(19) United States
(12) Patent Application Publication
(10) Pub. No.: US 2025/0312243 A1
(43) Pub. Date: Oct. 9, 2025

(54) CLOSED-LOOP ARCHITECTURE FOR DISTRIBUTING AND ADMINISTERING MEDICINES TO PATIENTS

(71) Applicants: REGENTS OF THE UNIVERSITY OF MICHIGAN, Ann Arbor, MI (US); SUBLIME, LLC, Okemos, MI (US)

(72) Inventors: Max Shtein, Ann Arbor, MI (US); Mohsen Moayer, New York, NY (US)

(21) Appl. No.: 18/860,543

(22) PCT Filed: Apr. 28, 2023
(86) PCT No.: PCT/US2023/020444

§ 371 Date: Oct. 25, 2024

Related U.S. Application Data
(60) Provisional application No. 63/336,195, filed on Apr. 28, 2022.

(51) Int. Cl. A61J 7/04; A61J 7/00; A61K 41/00; G16H 10/60; G16H 20/10

(52) U.S. Cl. CPC A61J 7/0418; A61J 7/0076; A61J 7/049; A61K 41/00; G16H 10/60; G16H 20/10

(57) ABSTRACT

The following relates generally to administering and delivering medications to patients. In some embodiments, an in-home appliance may place an ingestible ingredient (e.g., an active pharmaceutical ingredient (API) of a medication) on a physical substrate to create a distributable/retrievable object, which is distributed to a subject. The subject may then consume at least a portion of the ingestible ingredient, and then return at least a portion of the distributable/retrievable object to the appliance. The appliance, upon receiving the object, can subsequently trigger secondary actions, including logging the consumption of the dose: authorizing the next dose to override security measures which would otherwise block access to additional doses: analyzing the returned object for biometrics or remaining amount of ingestible ingredients, etc. Logged data will populate a software database on the cloud which can integrate with any number of internal and external systems, triggering programmable actions within a closed-loop architecture.

### CROSS-REFERENCE TO RELATED APPLICATIONS

This application claims priority to U.S. Provisional Application No. 63/336, 195, filed Apr. 28, 2022, which is incorporated herein by reference in its entirety.

### BACKGROUND

Existing systems and methods for distributing and administering medicines to patients in home settings constitute a fundamentally open-loop architecture which makes it impossible to accurately and comprehensively track patient dosing habits, in which individual doses, their amounts, and the time and amount ingested cannot be reliably tracked. Furthermore, conventional systems, particularly those designed for the home setting, are incapable of authenticating a patient prior to making a dose available, and cannot effectively restrict access to doses at higher amounts and frequencies than prescribed. These limitations of the existing open-loop architecture lead to poor adherence, overdosing on dangerous drugs, and associated negative patient outcomes including deaths, while also driving up healthcare costs.

In one simple example, the most common technique for medication administration is for a patient to take pills out of pill bottles. However, this has the drawback that there are no means of controlling how many pills the patient takes out, and no means of restricting unauthorized access.

Innovations from electronic internet-based pharmacies have led to delivery of pre-packaged doses, labeled with the time and day a given dose or combination of doses should be taken. However, this technique is still open-loop.

Another example technique, “smart” pill containers, which may be bottles or blister packs, offer a partial solution. For instance, some smart pill bottles have programmable timers to remind patients of their dose time and track when the container is breached (e.g., by the opening of a bottle or the puncturing of a blister pack). However, this solution assumes without the ability to verify that any breach of a container results in the ingestion of a dose, and that no more than one dose is ingested. Such systems cannot reliably control how many pills are extracted, cannot track what happens to any dose(s) once the container is breached, and offer no means of validating the user or restricting unauthorized access.

In another example, smartphone apps exist which offer the ability to track patient dosing behavior. However, these typically integrate with existing systems for administering medicines, such as smart pill bottles with a tracking app, without actually closing the loop. Such apps can only record data that a given architecture is already capable of generating. Alternatively, they require user/patient action by accessing the app through a smartphone to record their dosing activity—a tall order for a patient already at risk of non-adherence to their drug regimen.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, a method for tracking consumption of one of more ingestible ingredients may be provided. The method may comprise: determining, via one or more processors, a first dosage of an ingestible ingredient to administer to a subject; placing the determined first dosage of the ingestible ingredient onto a physical substrate to create a distributable object; distributing the distributable object to the subject; receiving at least a portion of the distributable object back from the subject; evaluating, via the one or more processors, the received at least a portion of the distributable object; and logging, via the one or more processors, the evaluation of the received at least a portion of the distributable object.

In another aspect, a system for tracking consumption of an ingestible ingredient may be provided. The system may comprise: a distributor device comprising a housing, and a communication interface; and a dispenser device housed within the distributor device, the dispenser device being configured to place dosages of ingestible ingredients onto physical substrates. The one or more processors may be configured to: determine a given dosage of an ingestible ingredient to administer to a subject; control the dispenser device to place the determined dosage of the ingestible ingredient onto a physical substrate to create a distributable object; control the distributor device to distribute the distributable object to the subject; control the distributor device to receive at least a portion of the distributable object back from the subject; evaluate the received at least a portion of the distributable object; and log the evaluation of the received at least a portion of the distributable object.

In yet another aspect, a system for tracking consumption of an ingestible ingredient may be provided. The system may comprise: a plurality of warehouses, each warehouse of the plurality of warehouses including: (i) a first section for storing cartridges comprising ingestible ingredients, and (ii) a second section for storing spent or partially spent cartridges; a plurality of vehicles configured to transport: (i) the cartridges comprising ingestible ingredients, and (ii) the spent or partially spent cartridges; a distributor device comprising a housing, and a communication interface; and a dispenser device housed within the distributor device, the dispenser device being configured to place dosages of the ingestible ingredients onto physical substrates. The system may further comprise one or more processors configured to: determine a first dosage of an ingestible ingredient to administer to a subject; control the dispenser device to place the determined first dosage of the ingestible ingredient onto a physical substrate to create a distributable object; control the distributor device to distribute the distributable object to the subject; control the distributor device to receive at least a portion of the distributable object back from the subject; evaluate the received at least a portion of the distributable object; and log the evaluation of the received at least a portion of the distributable object.

In yet another aspect, a method for tracking consumption of an ingestible ingredient may be provided. The method may comprise: determining, via one or more processors, a sequence of dosing events; distributing a first distributable object to a subject, the first distributable object including a first dosage of an ingestible ingredient specified by a first dosing event of the sequence of dosing events; and sequentially distributing subsequent distributable objects to the subject, wherein each distribution event of the sequential distribution: (i) occurs in response to receiving authorization based on receiving a previously distributed distributable object back from the subject, and (ii) is logged via the one or more processors.

### BRIEF DESCRIPTION OF THE DRAWINGS

FIG. 1A shows a system for tracking patient medication protocol adherence, in an example.

FIG. 1B shows a system for tracking patient medication protocol adherence, including additional components of the distributor device, in an example.

FIG. 1C shows a system for tracking patient medication protocol adherence, including delivery capabilities, in an example.

FIG. 2A illustrates a distributable object, in an example.

FIG. 2B illustrates a distributable object including an indicia, in accordance with another example.

FIG. 3 illustrates an example dispenser device.

FIG. 4 illustrates an example dispenser device including an activator and a detector.

FIG. 5 is a schematic of an example vapor deposition device, in accordance with an example.

FIG. 6 is a schematic of the use of the vapor deposition device of FIG. 5 to produce stocks of compounds in solution at a controlled concentration.

FIG. 7 is an example schematic of the use of the example vapor deposition device of FIG. 5 to produce deposit films of compound on a distributable object.

FIG. 8 is an example schematic of the use of the vapor deposition device of FIG. 5 to produce stage (A) discrete patterns of an ingestible ingredient on a distributable object, or stage (B) a continuous gradient of drug(s) across the distributable object.

FIG. 9 illustrates an example cartridge.

FIG. 10 illustrates an example secured receptacle.

FIG. 11 illustrates an example flowchart for tracking patient medication protocol adherence, including additional aspects of sequential dosing.

FIG. 12 illustrates another example flowchart for tracking patient medication protocol adherence.

FIG. 13 shows an example flowchart illustrating exemplary actions that may be taken in response to a comparison between the remaining amount of the ingestible ingredient, and a predetermined threshold.

FIG. 14 illustrates an example flowchart for actions relating to authentication and authorization.

FIG. 15A illustrates examples of open loop systems, in a prior art example.

FIG. 15B illustrates a closed loop system, in accordance with an example.

FIG. 15C illustrates a closed loop system, in accordance with another example.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

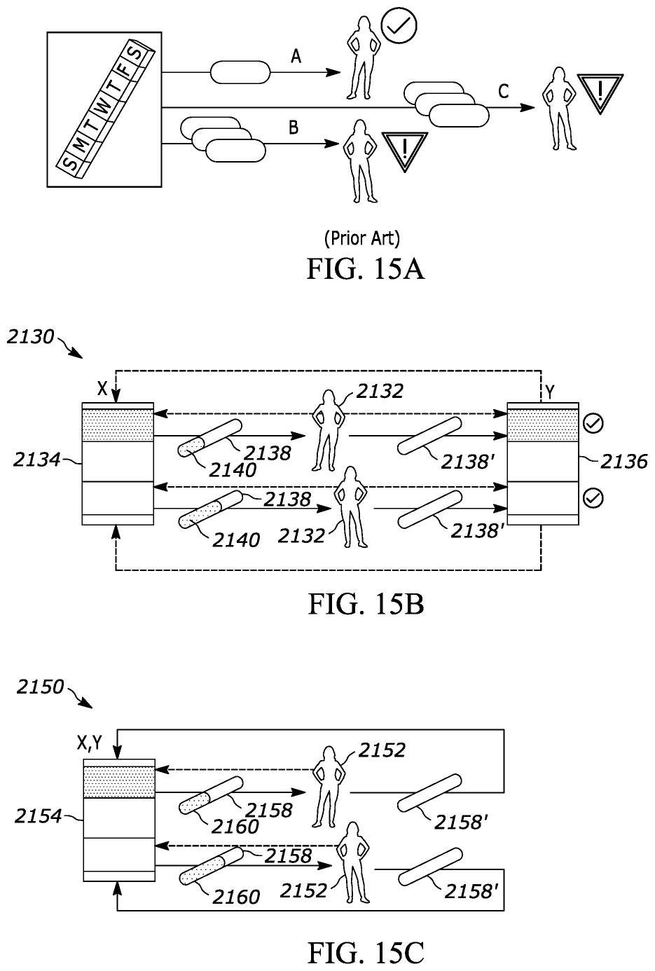

Current techniques for administering medications and tracking patient medication protocol adherence suffer are sorely lacking. Various entities have proposed pill bottles, pill blister packs, etc. that are purportedly smart, in that they are designed to administer a predetermined dosage, but do not perform actual tracking of what happens to a dose when it leaves a container, whether the dosage was taken or not or how much was taken, nor controlling the follow-on dose based on taking of the preceding dose. Consider a typical set of scenarios. First, a prescription is made by a doctor. Then, the patient receives entire course of medicine (e.g., in a pill bottle or blister pack). The patient then takes medicine as prescribed (e.g., case A of FIG. 15A). However, other outcomes are possible: For example, the patient can neglect to take the medicine. In another example, the patient can overdose on medicine (e.g., case B of FIG. 15A). In yet another example, the wrong person can take the medicine (e.g., case C of FIG. 15A). Existing systems are ‘open-loop’ in this manner and that hinders long term medication adherence and treatment efficacy.

One known process is as follows. Step 1: doctor sends the prescription to a pharmacy. Step 2: the pharmacy fills the prescription. Step 3: the patient retrieves the filled prescription from the pharmacy. Step 4a: the patient accesses the medicine from the container (e.g., pill bottle) at the prescribed time (or not). Step 4b: the patient takes the medicine according to prescribed amount (or not). Step 5: the doctor and other authorized parties learn (or not) whether the patient took (or did not take) their medicine according to the prescribed regimen, to better understand the efficacy of the prescribed medicine, to gain insight into under-/over-dosing, and to prevent potential negative outcomes (e.g., when the patient may be misusing a prescribed opioid). This sequence constitutes a feedback loop, however, under current practices it experiences failures at multiple points. For example, at Step 3, the patient may fail to retrieve the prescription. Online pharmacy services (e.g., PillPack) can help close this part of the loop through home delivery. At Step 4a, conventionally, there is not a way to ensure that the timing of the dose is correctly observed. Smart pill bottles, smart blister packs, and reminders from smartphone apps can help with remembering to take the medicine at the right time, and/or to log the time of access of the medicine. At Step 4b, however, none of these solutions track the dosage amount ingested by the patient. As a result, it is difficult for these solutions to fulfill the requirements of Step 5. While there are smartphone apps that purport to capture the required data, they require additional action on the part of the patient, who may already be at risk of poor adherence. Furthermore, if the patient does not complete the prescribed course (e.g., takes only 20 of the prescribed 30 pills), the remaining medicine can easily fall into the wrong hands—e.g., an opioid sold illicitly.

Alternative systems to the above include so-called smart pill dispensers. smart pill dispensers offer a partial solution, but their requirement to be manually loaded with pills by a patient or other household user creates a significant loophole in the preceding sequence, where any number of pills may be available to any party with access to the pills before they are loaded. Additionally, these dispensers do not offer security measures such as anti-tamper and self-destruct mechanisms.

Another alternative system is based on a “smart” or electronic pill, which sends a signal to a separate device, typically a wearable electronic patch, upon ingestion. However, such solutions may introduce potential safety risks to the patient, with no studies having examined the long-term health impact of ingesting the microelectronics contained in these types of pills, for example by the possibility of their accumulation in the gastrointestinal tract or other organs. Furthermore, the requirement to wear and replace a patch to receive transmitted information can create an added burden against adherence.

The devices, systems, and methods disclosed herein constitute a novel “closed-loop architecture” for distributing and administering drugs to patients, while removing certain demands made on the patient by alternative systems (such as the need to manually log dosing activity). The novel closed-loop architecture for distributing and administering drugs to patients disclosed herein can be the foundation of a digital health platform that can help solve the problems of drug overdosing and unauthorized access to medicines, non-adherence to drug regimens, and related problems.

In various examples, devices, systems and methods are provided for addressing the limitations of the approaches listed above, by dispensing and tracking consumption of an ingestible ingredient to a subject, in particular using a closed loop, secured approach. In some embodiments, a an appliance, such as a distributor device, registers a subject, identifies an ingestible ingredient to provide the subject and creates a distributable object with that ingestible ingredient applied on it, for dispensing to the subject. After the subject returns the dispensed distributable object, the appliance automatically analyzes the object, determines an amount of ingestible ingredient that was consumed, and assesses next steps. For example, the appliance may create and distribute a next distributable object, the appliance may notify a care provider of a subject's compliance or lack of compliance with a prescribed consumption protocol. Consider a scenario. Step 1: prescription is made. Step 2: the pharmacy fills the prescription in the form of the medicine enclosed in a secure cartridge, which will be described in greater detail below. Step 3: the patient receives home delivery of the cartridge and inserts it into an appliance (e.g., a distributor device, as will be described further below). Step 4a: the appliance reminds the patient at a prescribed time (with any combination of visual and/or auditory alerts on the appliance itself, alongside optionally alerting the user's or patient's electronic devices), and presents, to the patient, the correct dosage on a distributable object. Step 4b: the patient takes the distributable object containing the medicine in the prescribed amount and ingests the medicine from the object. Step 4c: the patient returns the object (with the medicine now removed) back to the appliance. Step 4d: the appliance logs the return event. Step 4e: the appliance populates a cloud-connected database with the associated return event information. Step 5: a system can access the database, offer real-time or delayed notifications to healthcare providers or otherwise authorized parties of whether the patient took (or did not take) their medicine according to the prescribed regimen; additionally, the system can offer analytics on captured data. By introducing this modified and automated sequence, involving a distributable and retrievable object carrying the medicine, the loop is closed, enabling authorized healthcare professionals to access patient adherence information in real-time and historically, and offering analytics at individual patient and population levels and points in between, while also enabling artificial intelligence and machine learning based predictive models of behavior. This inherently closed-loop architecture with anti-tamper and self-destruct capabilities also creates a system that can prevent overdosing. The new approach simplifies adherence for the patient, makes it easier to administer and improve prescribed regimens for doctors, reduces medicine waste, and reduces likelihood of misuse. The new sequence and hardware also enable the means of adjusting subsequent doses and/or introducing interventions based on the generated information about each dosing event, without waiting to finish the entire prescribed course. By involving a caregiver in the loop, the system can also collect additional information on the administration patterns of the caregiver.

In some examples, the closed-loop architecture herein also introduces sequential dosing. In current drug delivery and administration protocols, all doses within a container are equally accessible and generally assumed to have equivalent amounts of each ingredient (within a certain manufacturing tolerance). From a pill bottle or a blister pack, it makes no difference which pill a patient extracts, and typically, the patient can easily access any number of pills. This current architecture severely limits the ability to track and trace individual doses, and adjustments made to a patient's supply of medicines will lag behind any changes to their prescribed regimen, while generally wasting doses in the old amount. In contrast, the techniques described herein allow each dose to be tracked and traced individually via the system's closed-loop architecture. In this novel architecture, dose N+1 cannot be accessed until dose N has been taken, and, as an added security measure, access to dose N+1 may be prevented until the distributable/retrievable object from dose N was returned to the appliance's return receptacle. In one example, if the 8^{th} dose was missed and then the patient got back on track, the system will be able to track that the 8^{th} dose was taken at the scheduled time of the 9^{th} dose, and log this series of events while calculating the necessary adjustments to keep the patient in line with their prescribed regimen. In another example, if a patient is prescribed a highly addictive opioid such as oxycodone, the secured capability of the techniques described herein will not release the N+1 dose until the N dose has been ingested, and its distributable/retrievable object returned to the appliance's return receptacle. In this example, the anti-tamper and self-destruct security features may block access to doses out of sequence according to the prescribed schedule, within a specified tolerance, in cases where a clinician may want to allow a patient flexibility to access a limited increase in their dosing at the patient's discretion. In addition to overdose prevention, this combination of sequential dosing with the distributable/retrievable object also enables other novel features of this architecture, such as metering of doses, and the ability to, in real-time, adjust a patient's dosing regimen according to prescribed changes.

Some embodiments leverage distributed ledger technology to, for example, even further improve the tracking of consumption of an ingestible ingredient. In the current pharmaceutical supply chain, the blockchain could be deployed to track medicines from a manufacturing facility, to a distribution center, to a pharmacy, and potentially, to the point at which a patient receives a container of medicines (e.g., a pill bottle) from a pharmacy. However, in the existing architecture of delivering and administering doses to patients, blockchain's access to data is limited in two important ways. Firstly, the inability to track a container of medicines past pickup from a pharmacy makes it impossible to trace the rest of its path, i.e., the blockchain will be blind to whether the medicines were taken as prescribed, illicitly sold, left to expire, or fell into the hands of a child. Secondly, the existing architecture for distributing and administering medicines to patients is designed to track the pill container, but its open-loop nature limits the ability to track doses individually; therefore, data associated with individual dosing (e.g., timing, amount, authorization verification) is inaccessible to the blockchain without additionally burdening patients or caregivers, such as by manually logging doses, requiring the patient to wear a Bluetooth-connected patch, or otherwise increasing the number of steps and obligations to ensure the requisite data capture. Only by integrating with a closed-loop architecture for distributing drugs and administering them to patients, can the blockchain usefully track and trace dosing information while conferring its privacy and security advantages to confidential patient data.

In contrast to current practice, the closed-loop architecture of the techniques described herein enables the automatic creation of a transaction, which can be placed onto a distributed ledger, such as a blockchain, and used to track each dose of a drug product, from creation to ingestion, with the ability to flag whether it has been retrieved by an authorized user as scheduled. This closed-loop architecture for delivering and administering medicines to patients therefore may constitute a “Blockchain Oracle” for patient dosing data, which converts live patient dosing data into transactions, or contracts, which populate a distributed ledger, such as a blockchain. The information automatically generated by this closed-loop architecture can also serve as the data backbone for medical decision-making and predictive analytics enabled through artificial intelligence and machine learning algorithms.

In various examples, a system is provided to generate and dispense a distributable object. Generation may include, in response to authenticating a subject (e.g., a user and/or care professional), selecting an ingestible ingredient and applying it to physical substrate of a distributable object. A recipient (e.g., a patient or a caregiver) authenticates themselves as an authorized user of the dispensing module. The dispensing module produces a distributable object containing the dose and provides it to the recipient, which the recipient administers as prescribed. After administration, the distributable object is deposited into a receiver module, which logs (and optionally analyzes) the used distributable object. Relevant information about the distributable object (and optionally the user) is fed back (optionally via a network) to the dispensing module. In this manner, control over the dispensing of the follow-on dose via a distributable object is enabled. The user(s) interacting with the dispensing and receiving units is/are authenticated, and this information is used to determine the composition of the next distributable object. Note that the dispensing module X and the receiving module Y can be different devices, or they can be the same devices, or they can be housed within the same device. Note also that control of the dosing regimen is established based on feedback—the particulars of resulting adherence (and/or the medicine's action), enabling a kind of quality control over the regimen. This contrasts markedly with current practice of prescription events triggering many possible outcomes, many of which may be deleterious (e.g., patient inadvertently overdosing, pharmaceuticals being misused intentionally, etc.).

Herein, distributable object refers to a physical substrate onto which the ingestible ingredient is placed. As an example, the substrate may be a stainless steel tongue depressor. As another example, the substrate may have a U-shaped cutout at the end, holding the dose, in a form such as a pill, gel/gummy, or a film strip. As another example, the substrate may have a capacitance sensor at one end that senses the placement of the dose in the mouth with the lips closing and contacting the patient-facing end of the substrate. As another example, the substrate may have another capacitance sensor at the distal end, where the patient will hold the substrate. In another embodiment, these capacitance sensors may be replaced or accompanied by pressure sensors. In another embodiment, there will be a moisture sensing element at the patient-facing end of the substrate. Such objects that are dispensed by a secured system and contain an ingestible ingredient to be taken by a subject and that is to be returned to the secured system for data capture and optionally analysis. The subject consumes at least a portion of the ingestible ingredient and then returns the distributable object back to the secured system, which may then evaluate the returned distributable object to capture the time of, and optionally other data related to, the administration of the dose such as an amount remaining and/or consumed of the ingestible ingredient. Various techniques for automatically evaluating returned distributable object may be used. Example evaluation techniques include image analysis techniques, chemical composition techniques, weight analysis techniques.

Subsequent to the return of the distributable object to the appliance, based on the evaluation and/or any other factors (e.g., prescription information, nature of the ingestible ingredient, amount ingested relative to a specified threshold, dosage timing, recommendations of physicians, etc.), the system may determine a second amount of the ingestible ingredient to distribute to the subject. Thus, such embodiments advantageously produce a closed-loop system. That is, by receiving the remaining portion of the distributable object back from the subject, constructive feedback may be created, and used in determining future amounts of the ingestible ingredient to distribute to the subject.

As used herein, an ingestible ingredient is an active pharmaceutical ingredient (API), an excipient, a placebo, a flavoring, a dietary supplement, or a combination thereof. Examples include tramadol, oxycodone, fentanyl, acetaminophen, ibuprofen, tamoxifen, palbociclib, letrozole, vitamin D3, ketamine, nicotine, cannabidiol, vanillin, erythritol, pullulan, microcrystalline cellulose, lactose, polylactic acid, folic acid, calcium citrate, ferrous sulphate, and fiber supplements. Further APIs may include various drugs or potential drugs (e.g., new chemical entities), including anti-proliferative agents; anti-rejection drugs; anti-thrombotic agents; anti-coagulants; antioxidants; free radical scavengers; nucleic acids; saccharides; sugars; nutrients; hormones; cytotoxins; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; acetylcholinesterase inhibitors; and combinations thereof. The description of these suitable organic compounds/pharmaceutical active ingredients/new chemical entities is merely exemplary and should not be considered as limiting as to the scope of compounds or active ingredients which can be applied to a surface according to the present disclosure, as all suitable organic molecules and/or active ingredients known to those of skill in the art for these various types of compositions are contemplated. Furthermore, an organic or inorganic compound may have various functionalities and thus, can be listed in an exemplary class above; however, may be categorized in several different classes of active ingredients.

### Example System

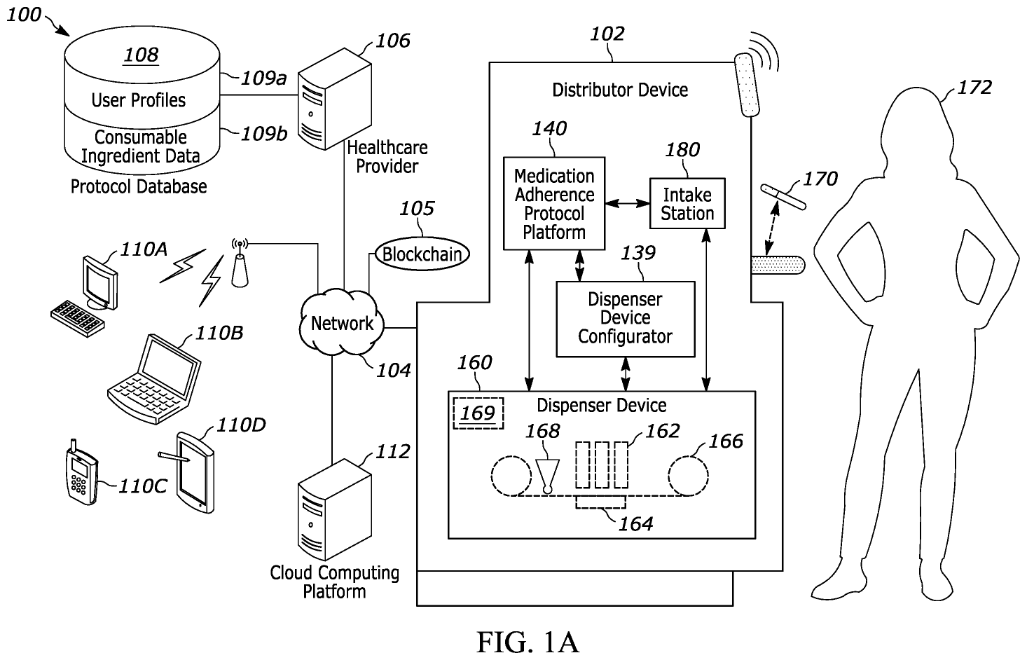

FIG. 1A illustrates an example system 100 for tracking consumption of an ingestible ingredient. The system 100 includes a distributor device 102 capable of generating a distributable object 170 having an ingestible ingredient 162 and dispensing the object 170 to a subject 172 for consumption of the ingestible ingredient. The system 100 provides closed loop subsequent tracking of the subject's 172 consumption of the ingestible ingredient and/or adherence to a medication protocol.

To this end, the illustrated example includes medication adherence protocol platform 140. Broadly speaking, the medication adherence protocol platform 140 may be used to determine doses of the ingestible ingredient 162 to be place on a physical substrate 164 which may be applied to the distributable object 170 or which may be portion thereof, such as an outer surface. For example, the subject 172 may return the used distributable object 170 to the distributor device 102 at intake station 180 or the dispensary door 182. The distributor device 102 may then evaluate the returned distributable object 170 to determine a remaining portion of an ingestible ingredient 162. Based on the evaluation, the medication adherence protocol platform 140 may then determine future doses of the ingestible ingredient 162 to distribute to the subject 172.

For instance, the medication adherence protocol platform 140 may determine future doses based on the retrieved distributable object after administration, and optionally on an amount of the ingestible ingredient remaining determined during the evaluation. In one example, if all of the ingestible ingredient has been consumed, the medication adherence protocol platform 140 may determine the next dosage to be a recurring amount of the ingestible ingredient. In another example, if there is more than a predetermined amount of ingestible ingredient remaining, the medication adherence protocol platform 140 may modify a next dosage of the ingestible ingredient based on the amount remaining, information in a user profile of the subject, etc.

Moreover, the medication adherence protocol platform 140 may log information of the evaluations in the subject's user profile (e.g., stored as user profile data 109a in protocol database 108), thereby creating a record of the subject's adherence to protocols for taking medication or other ingestible ingredients, as will be described further below.

The distributor device 102 may be a secured, closed loop system and includes a dispenser device 160 for presenting the distributable object 170 to a user, who may be a caregiver or a patient. Prior to presenting the distributable object 170, the object 170 is selected from a series of substrates previously combined with an ingestible ingredient containing the dose, in a form such as a pill, gel/gummy, or a film strip, affixed onto one end. The combining of the substrate with the ingestible ingredient may occur at a separate manufacturing facility or an automated pharmacy designed according to the systems and prescription-management protocols described in this invention. Alternatively, the dispenser device 160 may fabricate the ingestible ingredient 162 onto the physical substrate 164 to create the distributable object 170. The ingestible ingredient 162 may be determined by the medication adherence protocol platform 140. As an example, for treatment of chronic conditions, during the course of which no changes in dosage are anticipated, the combining of the substrate with the ingestible ingredient may be preferably done at a separate manufacturing facility or an automated pharmacy. In a use case where active management of a condition is anticipated, such as a dose-tapering regime for an opioid drug, the combining of the substrate with the ingestible ingredient may be preferably done inside the dispenser device.

The dispenser device 160 may be configured into variety of different fabrication types to form the ingestible ingredient on a physical substrate. Various fabrication modalities include lamination, 3D printing, spray coating, dip coating, vacuum coating, sputtering, ink-jet printing, dip-pen writing, etc. In some examples, the dispenser device 160 includes rollers 166 and other mechanical components 168. The dispenser device 160 may unspool the ingestible ingredient 162 from a section of tape in a cartridge to place the ingestible ingredient 160 onto the physical substrate 164.

The dispenser device 160 may further include controller 169 having one or more processors. The controller 169 may control the rollers 166 and/or mechanical components 168 to place the ingestible ingredient 162 on the physical substrate 164 (e.g., in accordance with instructions sent from the dispenser device configurator 139, etc.). Another example function of the controller 169 is monitor remaining levels of the ingestible ingredient 162. For example, if the ingestible ingredient 162 in any source (e.g., a cartridge with a spooled tape of the ingestible ingredient) falls below a predetermined level, the controller 169 may provide an alert (e.g., in the form of a signal to the one or more processors 115 of the distributor device 102 illustrated in the example of FIG. 1B) indicating that an amount of the ingestible ingredient 162 is low, and unlock the source (e.g., cartridge, etc.) so that the depleted source may be replaced with a full source. In this regard, following receipt of the alert, the distributer device 102 may display an alert on the display 126 indicating the low ingestible ingredient level. Additionally or alternatively, the distributor device 102 may send an alert (e.g., in the form of a signal) to the warehouses 190 of the example of FIG. 1C, alerting the warehouses 190 of the low ingestible ingredient level.

The distributor device 102 may be coupled to a network 104 for receiving and communicating various types of data. The network 104 may be a wireless or wired network communicating with various computing devices, including, in the illustrated example, a healthcare provider computing system 106 having a medication protocol database. The healthcare provider computing system 106 may represent a physician's computing terminal at a hospital or other healthcare facility. The protocol database 108 may store medication data for a plurality of active or inactive ingredients (such as AIPs or supplements) to be used in treating a subject 172, in particular active ingredients available for oral delivery to a patient. The protocol database 108 may store user profiles 109a. The user profiles 109a may include data of: an age of the subject, a gender of the subject, a weight of the subject, a height of the subject, body mass index of the subject, genetic information of the subject, preferred place of medication administration, preferred manner of administration, preferred foods of the subject, counter-indicated foods, person responsible for administration, address of the subject, service subscription information, service renewal information, criminal history of the subject, etc.

The protocol database 108 may further optionally store (as part of the user profiles 109a, or separately from the user profiles 109a) ingestible ingredient data 109b including: molecular weight of an API, chemical structure, chemical database reference number(s), solubility, enthalpy of vaporization, vapor pressure, melting point, thermal decomposition point, viscosity, hardness, taste profile, counterindications, co-prescriptions, dosage, expiration dating information, storage instructions, safety instructions, associated billing codes, prescribed regimen for a given patient, regimen starting time and date, regimen ending time and date, administration restrictions (e.g. temperature sensitive APIs restricted from certain physical substrates), etc. As discussed in examples herein, a healthcare professional may provide any of the data from the medication protocol database 108 to the healthcare provider system 106 for provision to the distributor device 102.

Further in the example of FIG. 1A, various patient devices (e.g., computing devices of the patient or subject 172) are also connected to the network 104 through which patients can enter identification data to the distributor device 102. The patient devices may present an instantiation of an accessing (app) to provide such identification data and to allow the patient devices to be individually authenticated from communication with the distributor device 102. In the illustrated examples, patient devices include a computing terminal 110A, a laptop computer 110B, a mobile cellular device 110C, and a mobile tablet computing device 110D. Other patient devices include personal mobile and/or monitoring devices, such as smart watches and other wearable monitoring devices, such as heart rate monitors. Of course, provided devices may be coupled to the distributor device 102 through the network 104 and these provided devices may be computer terminals, laptop computers, mobile cellular devices, mobile tables, and the like, as well.

Further, as shown, a cloud computing platform 112 is connected to the distributor device 102 through the network 104. The cloud computing platform 112, as discussed herein, may receive data (including any data held by the distributor device 102) from the distributor device 102 and generate plans for distributable objects, including layered structures formed and patterned to provide medication to a patient. For example, although, in some embodiments, the medication adherence protocol platform 140 determines an amount of ingestible ingredient 162 to place on the substrate 164, in other embodiments, the cloud computing platform 112 determines the amount of ingestible ingredient 162 to place on the substrate 164. For instance, the cloud computing platform 112 may receive data including evaluation data of a returned distributable object 170, and calculate a next dose of the ingestible ingredient 162 to place on the substrate 164 based on the evaluation. Furthermore, the cloud computing platform 112, in some embodiments, may provide for or aid in the automated tracking of the amount of medication consumed by the patient. It may be noted that many calculations/computations/determinations as discussed herein may be performed at either the distributor device 102 or the cloud computing platform 112.

The network 104 may be a public network such as the Internet, private network such as research institution's or corporation's private network, or a distributed ledger such as a blockchain, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

FIG. 1B shows the example system 100 for tracking patient medication protocol adherence, but also illustrates additional example components of the distributor device 102 not illustrated in the example of FIG. 1A. In the example of FIG. 1B, the distributor device 102 includes one or more processors 115, one or more optional graphics processing units 116, a local database 118, a computer-readable memory 120, a network interface 122, and Input/Output (I/O) interfaces 124 connecting the distributor device 102 to a display 126, user input device 128, and/or camera 129.

The memory 120 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the distributor device 102 may represent a CPU-type processing unit, a GPU-type processing unit, a field-programmable gate array (FPGA), another class of digital signal processor (DSP), or other hardware logic components that can be driven by a CPU.

In the illustrated example, in addition to storing operating system 138, the memory 120 stores a medication adherence protocol platform 140, configured to execute various processes described and illustrated herein. In an example, the medication adherence protocol platform 140 includes a medication protocol platform 142 and an distributable object configurator 144, each in accordance with example techniques described herein. Additionally, the memory 120 includes an adherence protocol resolver 146 and plurality of different databases, in this example, a patient data database 148, a distributable object database 150, and a medication data database 152.

In some embodiments, the systems and methods described herein are designed for the automated data capture of each dosage event, whether the patient misses or ingests a dose, thereby creating a transaction event that is automatically capturable on a blockchain.

The blockchain 105 may be a network wherein participating network nodes (e.g., other network participants) validate changes to a distributed ledger based upon transactions, such as events sent from the medication protocol platform 142, broadcast by other network participants. In some embodiments, the other network participants of the blockchain include the distributor device 102, the healthcare provider 106, the cloud computing platform 112, the computing terminal 110A, the laptop computer 110B, the mobile cellular device 110C, and/or the mobile tablet computing device 110D. In addition, the blockchain 105 may be configured to store and execute smart contracts.

Further illustrated in the example of FIG. 1B is a dispensary door 182, which may be used, for example, to distribute the distributable object 170 to the subject 172. Also illustrated is an intake station 180. The intake station 180 may be used to receive the ingestible ingredient for creation of the distributable object 170. For example, a user may place cartridges containing the ingestible ingredient(s) into the intake station 180 to be used in the creation of the distributable object 170. In some implementations, the cartridges are first placed in medication dispenser apparatus 183, and then the medication dispenser apparatus 183 is placed into the intake station 180. Furthermore, either the dispensary door 182 or the intake station 183 may be used to receive distributable objects returned by the subject (e.g., distributable objects that the subject has consumed at least a portion of the ingestible ingredient of).

### Example System With Delivery Capabilities

FIG. 1C shows a system 100 for tracking patient medication protocol adherence, including delivery capabilities, in an example. With reference thereto, warehouses 190 may store, for example, the ingestible ingredients 164, cartridges containing the ingestible ingredients, the medication dispenser apparatuses 183, the physical substrates 164, distributor devices 102, components for repair of the distributor devices 102, etc. Furthermore, although the example of FIG. 1C illustrates only two warehouses 190, any number of warehouses may be used.

In some embodiments, the warehouses 190 have separate sections for storing separate components. For example, a warehouse 190 may have a first section for storing the ingestible ingredients 162, and a second section for storing the physical substrates 164. In another example, the warehouses 190 have a first section for storing ingestible ingredient cartridges ready to be transported from the warehouse 190 (e.g., full and unexpired cartridges), and a second section for storing cartridges not suitable for transport (e.g., cartridges received back after being fully or partially spent, expired cartridges, etc.). Advantageously, storing the cartridges in separate sections helps reduce the risk of inadvertently transporting the wrong cartridge or group of cartridges. Further advantageously, this partitioning of separate section allows for the appropriate equipment to be better located. For instance, the equipment that refills cartridges may be better located close to the section of the warehouse storing fully or partially spent cartridges.

Items may be transported to or from the warehouses by any technique. For instance the delivery truck 192, delivery car 194, and/or drone 196 may deliver items to or from the warehouses 190. In this regard, the items may be transported between the warehouse and the building 103, which may house the distributor device 102. The building 103 may be any kind of building holding the distributor device 102. For example, the building 103 may be a physician's office, a hospital, an urgent care facility, a retirement home, a personal home, an apartment building, a medical administration building, assisted living facility, a healthcare facility, etc.

In addition, the cartridges may be placed in medication dispenser apparatuses 183 (e.g., for storage at the warehouses 190 and/or transportation).

In some embodiments, the items to be transported are the distributor devices 102 themselves. For example, a distributor device 102 may be brought from the building 103 to the warehouse 190 for maintenance.

### Example Distributable Object

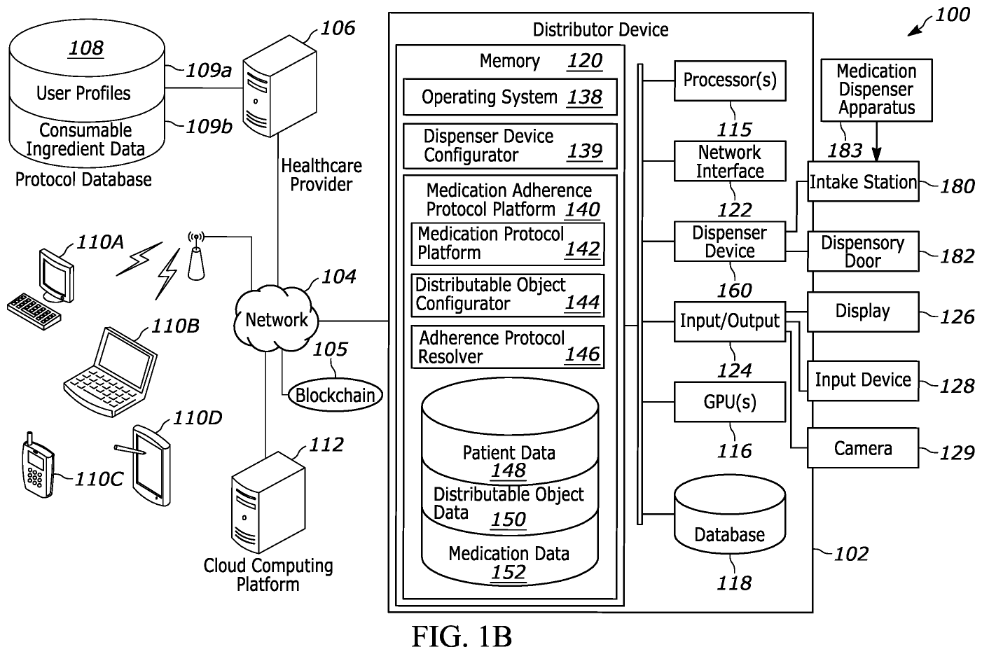

FIG. 2A illustrates an example distributable object 270 including a physical substrate 264 that is generally oval and flat (e.g., in the shape of a popsicle stick). However, it should be understood that this is only an example, and the physical substrate 264 may be any shape, size, roughness, etc. The example distributable object 270 further includes ingestible ingredient 262. As an example, the substrate 264 may be a stainless steel tongue depressor. As another example, the substrate 264 may have a U-shaped cutout at the end, holding the ingestible ingredient, in a form such as a pill, gel/gummy, or a film strip.

In some embodiments, the distributable object 270 includes sensors 266, 268, such as capacitance sensors, pressure sensors, moisture sensors, or combinations thereof. In one example, the sensor 266 is a capacitance sensor at one end that senses the placement of the dose in the mouth with the lips closing and contacting the patient-facing end of the substrate 264. In another example, the substrate 264 may have another capacitance sensor 268 at the distal end, where the patient will hold the substrate. Other examples include pressure and/or moisture sensors. For example, each of the sensors 266 and 268 illustrated in the example of FIG. 2A may individually represent any combination of capacitance sensors, pressure sensors, and/or moisture sensors.

FIG. 2B illustrates another example distributable object 280 having an ingestible ingredient 282 formed thereon. The distributable object 280 includes an indicia 281, such as a barcode, quick response (QR) code, a pattern, a color, etc. In some examples, the indicia 281 is positioned between the ingestible ingredient 282 and a substrate 284 and initially completely covered by the ingestible ingredient 282 upon object distribution by an appliance, such as the distributable device 102. The indicia 281 may be completely covered by the ingestible ingredient 282 upon distribution by the appliance or a partially covered. Advantageously, in various examples, the indicia 281 can be read by camera 129 when the object 280 is returned to the device 102, e.g., when the ingestible ingredient 282 has been completely or partially removed, namely by the recipient consuming the ingesting the ingredient 282. In some examples, an indicia 281 is of a chosen type and is positioned to provide a categorical output indicating of whether the ingestible ingredient 282 has been consumed, for example, indicating to a process connected to the camera 129 “consumed,” “not consumed,” or “partially consumed.” In some examples, the indicia 281 is of a chosen type and is positioned such that a processor connected to the camera 129 is able to assess captured image data and determine a continuous output indication, for example, indicating a percentage of ingestible ingredient 282 remaining and/or removed.

### Example Dispenser Devices

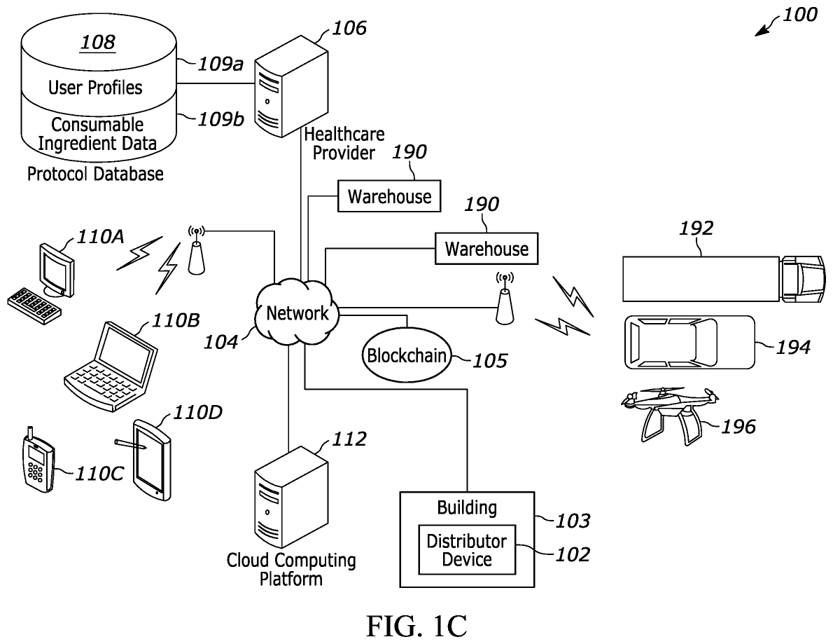

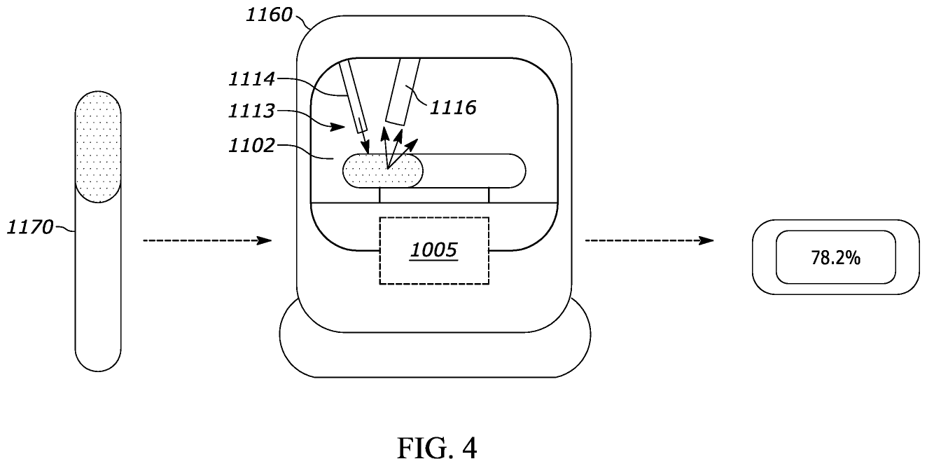

FIG. 3 illustrates an example dispenser device 1060 for dispensing an object having an ingestible ingredient. With reference thereto, a clean distributable object 1070a may be sent to the dispenser device 1060. The dispenser device 1060 may then house the distributable object 1070b in a sample area 1002, and place ingestible ingredient on the distributable object 1070b according to any of the techniques described herein. For instance, the depositors 1040 may be used to deposit drugs in vapor form, as is described with respect to FIGS. 5-8, or in another example, by laminating the ingestible ingredients. Following deposition, the distributable object 1070c may be removed.

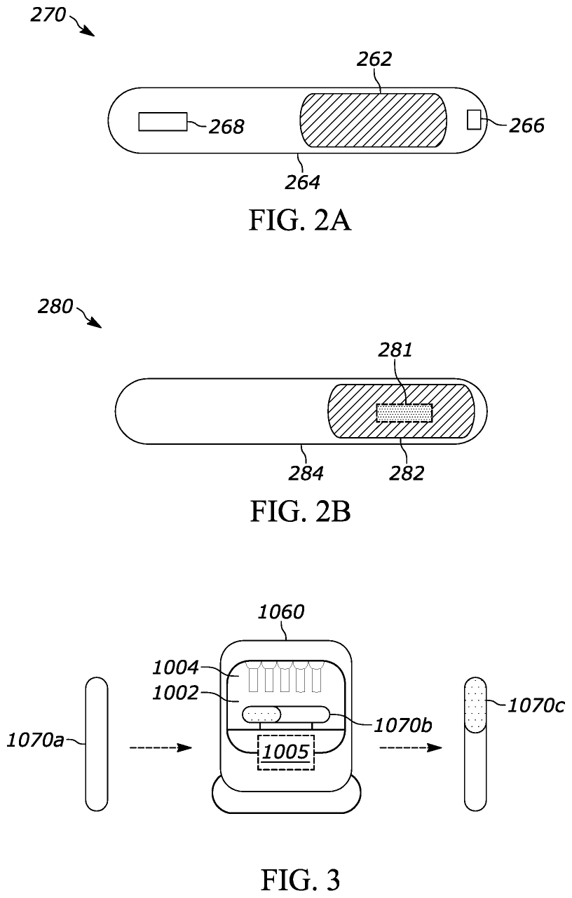

FIG. 4 illustrates an example dispenser device 1160, which may be used, for instance, for detecting an amount of ingestible ingredient remaining on the distributable object 1170. The dispenser device 1160 includes a sample area 1102 configured to hold the distributable object 1170, for example, using a mount in the sample area 1102 configured to hold the distributable object 1170.

An activation stage 1113, including activator 114 and detector 1116, may be positioned above the sample area 1102 to examine the distributable object 1170 and detect an amount of ingestible ingredient removed from and/or remaining on the distributable object 1170. In an example, the activation stage 1113 is an illumination activation stage for illuminating the distributable object 1170 in the sample area 1102 with a detection illumination. The activation stage 1113 may include a photodetector 1116 for detecting reflected illumination and generating a usage indication signal. In this regard, it should be understood that the activation stage 1113 may form a camera.

In another example, the activation stage 1113 is an ultra-violet (UV) illumination activation stage for illuminating the distributable object 1170 in the sample area 1102 with UV illumination. The activation stage 1113 may include a photodetector for detecting reflected UV illumination and generating a usage indication signal. In this regard, it should be understood that the activation stage 1113 may form an ultraviolet (UV) camera.

In another example, the activation stage 1113 is a thermal activation stage for applying a temperature increase in the sample area 1102. In this example, the dispenser device 1160 may include a thermal detector for detecting the usage indication signal.

In some examples, the dispenser device 1160 includes imaging equipment, such as having one or more cameras or UV cameras (e.g., formed of the activation stage 1113, or the activation stage comprising activator 1114, and detector 1116) with a corresponding field of view that includes the sample area and captures images of the sample area, in particular images or UV images of augmented distributable object 1170 or groups of augmented distributable objects 1170. The captured images or UV images may be analyzed, by the distributor device 102 or cloud computing platform 112, to determine an amount of ingestible ingredient consumed, remaining, or a combination thereof. In some examples, the distributor device 102 or cloud computing platform 112 may use a pattern recognition module to assess the captures images to determine an amount of ingestible ingredient consumed, remaining, etc. In some examples, that pattern recognition module may be configured with trained classifiers developed using a machine learning framework, such as classifiers configured as a convolutional neural network.

In addition, the dispenser devices 1060, 1160 may include scales 1005, 1105, which may be used to weigh the distributable object. The weight may then be used to determine the remaining amount of the ingestible ingredient based on, for example, a known weight of the physical substrate. In this regard, it should be understood that the remaining amount may be determined as a weight, or may be converted to a volume based on known characteristics of the ingestible ingredient. Moreover, the weight from the scale 1060, 1160 may be combined with information from the image or UV image discussed above to produce an even more accurate evaluation of the at least a portion of the distributable object. For example, the information from the image, and the weight may be input into a trained machine learning algorithm to determine a remaining amount of the ingestible ingredient.

The dispenser devices 1060, 1160 may also sterilize a distributable object once the subject returns it. For example, the dispenser devices 1060, 1160 may heat the distributable object to a predetermined temperature for a predetermined time period. In another example, the dispenser devices 1060, 1160 may apply UV radiation (or any other kind of suitable radiation) at a predetermined intensity for a predetermined time period. In another example, the dispenser devices 1060, 1160 may apply a gas to sterilize the distributable object. In yet another example, the dispenser devices 1060, 1160 may use a fluid (e.g., act similarly to an automatic dishwasher) to clean and/or sterilize the distributable object. Furthermore, any of these sterilization and/or cleaning techniques may be combined with each other or other techniques.

### Vapor Deposition Examples

FIG. 5 illustrates a schematic of an example vapor deposition device that may be used as part of the distributor device 102 (e.g., as part of the intake 180), in accordance with an example. An ingestible ingredient vapor may be jetted into a reservoir (left) with drugs and/or with solvents, wherein the compound vapor dissolves (right). A valve allows controlled introduction of the dissolved compound into a liquid handler, which can allow the compound to be introduced onto a distributable object.

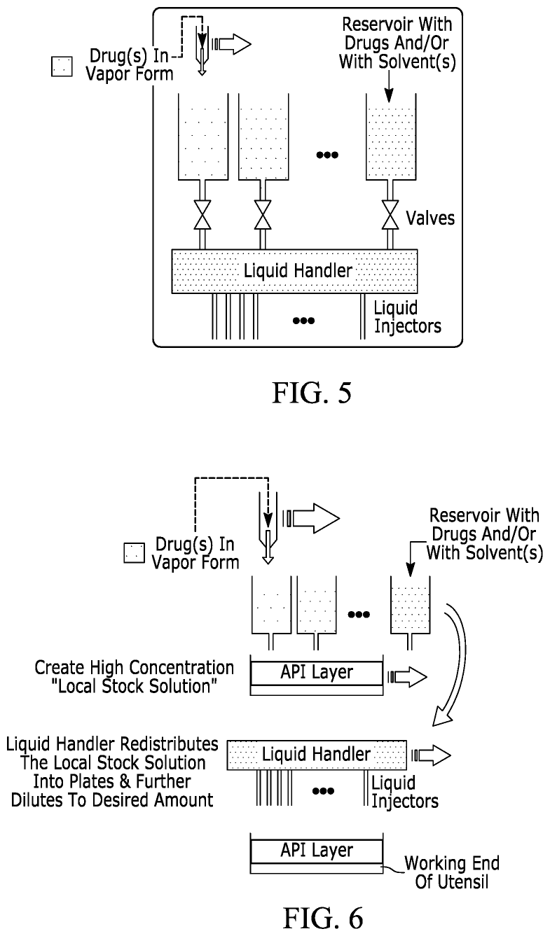

FIG. 6 is a schematic of the use of the vapor deposition device of FIG. 5 to produce stocks of compounds in solution at a controlled concentration. Ingestible ingredient vapor (e.g., API vapor, etc.) may be jetted into a reservoir (left) with drugs and/or with solvents, wherein the ingestible ingredient dissolves (right). This produces a high concentration “local stock solution” which can be redistributed by a liquid handler into well plates and further diluted to a desired concentration.

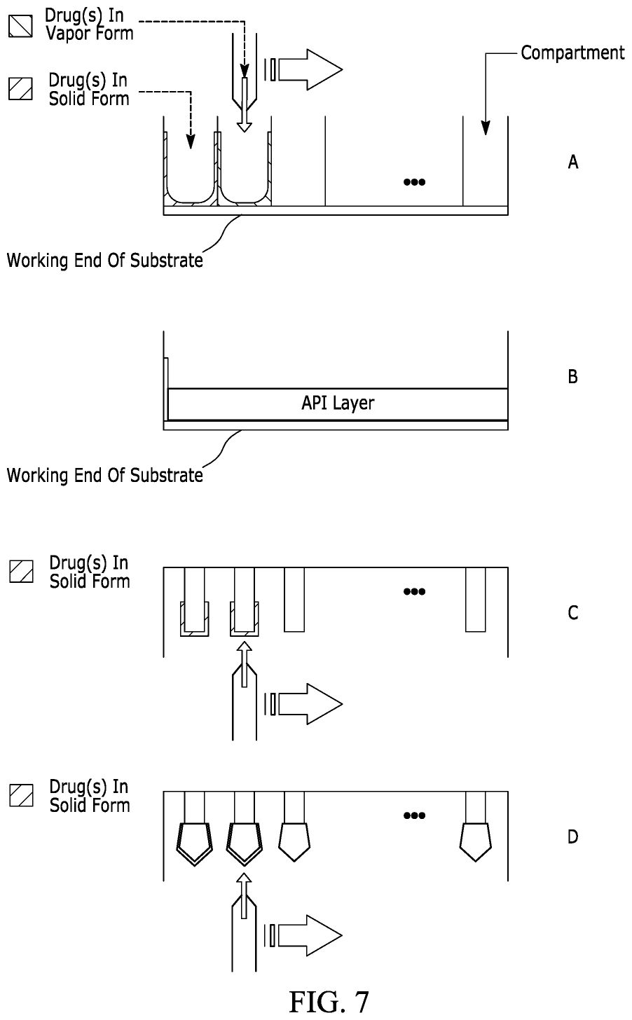

FIG. 7 is a schematic of the use of the vapor deposition device of FIG. 5 to produce deposit films of compound on a distributable object. With reference to FIG. 7, at stage (A), ingestible ingredient vapor (e.g., an API in vapor form) is jetted into a compartment to produce a solid film. At stage (B), the ingestible ingredient vapor can also be jetted directly onto the surface of the distributable object to form a film. At stage (C), ingestible ingredient vapor can also be deposited on a post to produce a solid film on the post. At stage (D), drug vapor can also be deposited on crystals or as part of a crystal growing process.

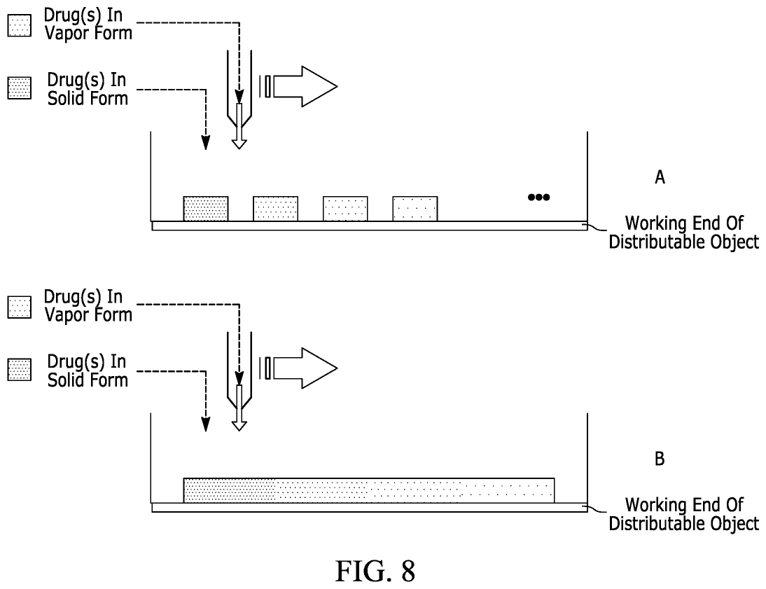

FIG. 8 is an example schematic of the use of the vapor deposition device of FIG. 5 to produce (A) discrete patterns of an ingestible ingredient (e.g., an API, etc.) on a distributable object, or (B) a continuous gradient of drug(s) across the distributable object.

### Cartridge Example

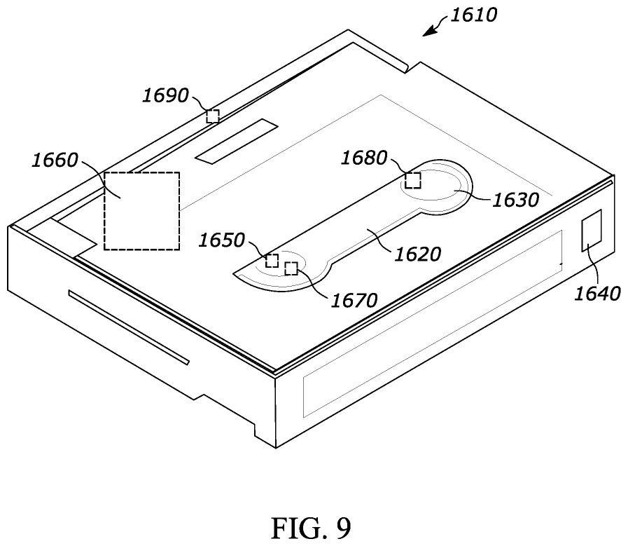

FIG. 9 illustrates an example cartridge 1610. In some embodiments, the cartridge 1610 contains a tape 1620 that contains the ingestible ingredient. In some implementations, the ingestible ingredient on the tape 1620 comprises a flavored pullulan film with API dispersed uniformly throughout, with a non-sticky or partially adherent backing (e.g., similar to a fruit roll-up). In some embodiments, the backing comprises a waxed paper backing.

The tape 1620 may have markings at regular intervals to enable metering a desired amount, using a mechanical counter, an optical counter, and/or a capacitive counter. In one example usage, the ingestible ingredient (or API) is levothyroxine, where most common dosage amounts can be achieved in ˜2.5 μg increments.

In another embodiment, the backing of the tape 1620 is inert and is dispensed along with the ingestible ingredient-containing film. In another embodiment, the backing of the tape is inert and is spooled up inside the cartridge 1610, while the ingestible ingredient-containing film may be spooled out of the cartridge 1610. In another embodiment, the backing of the tape 1620 may be adhesive, enabling lamination onto a secondary substrate (e.g., stainless steel tongue depressor or popsicle stick).

The tape 1620 with backing may be wound onto a spool 1630 made of inert material; the spool 1630 may be mounted inside the cartridge 1610; the spool 1630 advantageously adds dimensional and positional stability to the rolled-up tape 1620 and, optionally, enables coupling to a drive mechanism. In another embodiment, the spool 1630 may be triggered to deactivate the API (e.g., if the ingestible ingredient comprises an API). For instance, the API may be deactivated upon tampering with the cartridge. One method of deactivation is by release of heat from a heating element 1670 (e.g., a thermal battery, a resistively heated element, etc.) in the spool 1630. Another method of deactivation is by release of a reactive gas from a gas cartridge 1680 inside the spool 1630, housing the source of gas behind a pressure-sensitive membrane.

The detection of the tampering may be accomplished mechanically, chemically, electrically, and/or electronically. In the latter case, for example, the cartridge 1610 must receive contact or signal from the dispenser device in order to activate the spool 1630 and/or the door 1640. In some embodiments, not including such a signal, an internal contact sensor or fuse may be triggered upon opening of the cartridge or activation of the spool, logging an “event” in a memory of the processor(s) 1640 inside the cartridge 1610.

The spool 1630 is immobilized, until its rotation is unlocked, for example, by a locking mechanism 1650. In some embodiments, the locking mechanism 1650 is unlocked by engaging a lock mechanism with a key. In some embodiments, the locking mechanism 1650 is unlocked only by sending a secure pattern of signals to a connected electrical circuit. In some embodiments, the locking mechanism 1650 is unlatched by sending a pattern of signals wirelessly (e.g., by inductive coupling) at a predetermined frequency to an internal antenna-connected circuit. In some embodiments, the coincidence of two or more of the unlatching actions must be present to fully unlock (e.g., by unlatching) the spool 1630 to allow the spool 1630 to rotate.

The tape (e.g., with the ingestible ingredient) is dispensed through a cartridge door 1640, which is unlatched simultaneously with the rotation of the spool 1630. The cartridge door 1640 otherwise seals the cartridge 1610. The cartridge body may also contain a seal that reduces ingress of oxygen and moisture; the cartridge may optionally contain an absorbent material (e.g., a desiccant) to maintain low humidity.

The cartridge 1610 may also comprise one or more cartridge processors 1660, which may be used for any suitable purpose. For example, the one or more cartridge processors 1660 may control the locking mechanism 1650 (e.g., to latch or unlatch), and/or control the cartridge door 1640 (e.g., to open or close). However, these example functions may be controlled by any other technique (e.g., a mechanical technique to open the locking mechanism 1650, or controlled by other processors, such as the one or more processors 115).

The cartridge 1610 may optionally contain a wireless chip 1690. In some embodiments, the one or more cartridge processors 1660 control the wireless chip 1690 to report a location of the cartridge, a status of the cartridge, an access history of the cartridge, a temperature of the cartridge, and/or identifying information of the cartridge, such as reporting to the distributor device 102, the warehouses 190, the truck 192, the car 194, the drone 196, the cloud computing platform 112, and/or the healthcare provider 106.

The cartridge 1610 may be opened for examination, cleaning, repair, and/or recharging purposes upon appropriate activation, which may include a combination of authorization signals, mechanical unlocking (e.g., with a master key), physical location, and/or environmental conditions. The cartridge 1610 may also contain visible identifying information, instructions for use and/or return.

In some embodiments, rather than contain a tape 1620, the cartridge 1610 contains pre-prepared distributable objects (e.g., distributable objects with the ingestible ingredient already placed on it).

### Examples of Secured Receptacles

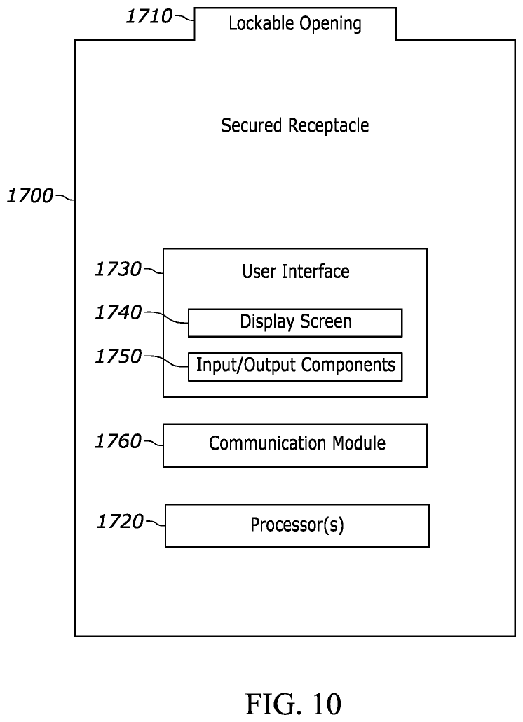

FIG. 10 illustrates an example secured receptacle 1700. The secured receptacle 1700 may have a lockable opening 1710, such as a lid or a door. In some embodiments, the distributable object is inserted by the distributor device into the secured receptacle 1700 through the lockable opening 1710.

The secured receptacle 1700 may also include a user interface 1730, including, for example, a display screen 1740, and/or I/O components 1750. In some embodiments, the user interface 1730 may display information about the a distributable object inside the secured receptacle 1700. The displayed information may include, for example, a type and/or amount of ingestible ingredient in the distributable object. A date and/or time for a subject to receive the distributable object, such as a date and/or time prescribed by a physician. Additional examples of information displayed include information of a caregiver distributing the distributable object. For instance, if a nurse receives the secured receptacle 1700 from the distributor device, and brings the secured receptacle 1700 to a patient, the display screen 1740 may display information of the nurse, such as the nurse's name, credentials, etc.

The I/O components 1750 may be any suitable components, and used for any purpose. For instance, the I/O components may be a touch screen keypad allowing a subject to enter a code (e.g., authorization information) that unlocks the lockable opening 1710. In another example, the I/O components 1750 comprise a fingerprint reader that allows a user to unlock the lockable opening 1710. In yet another example, the I/O components 1750 may comprise a camera that uses a facial recognition technique to unlock the lockable opening 1710.

It may be noted that in some embodiments, the I/O components 1750 are part of the display screen 1740, whereas in other embodiments, the I/O components 1750 are not part of the display screen 1740. For instance, in one example of the I/O components being part of the display screen 1740, the I/O components 1750 may comprise a touch screen having numbers and/or letter, thereby allowing a user to enter a passcode or password to open the lockable opening 1710. In an example where the I/O components 1750 are not part of the display screen 1740, the I/O components may comprise physical buttons.

The secured receptacle 1700 may further include communication module 1760. The communication module 1760 may be used for communication with the network 104. Additionally or alternatively, the communication module 1760 may communicate directly with other devices (e.g., by Bluetooth, etc.), such as other secured receptacles 1700, distributor devices, etc.

The secured receptacle 1700 may further include one or more processors 1720, which may also be referred to as one or more secured receptacle processors, or one or more processors of the secured receptacle. The one or more processors 1720 may perform any functions, such as receiving information from the I/O components 1750, controlling any of the components on the secured receptacle 1700 (e.g., the user interface 1730, the display screen 1740, the I/O components 1750, the communication module 1760, the lockable opening, etc.), etc.

### Example Methods

The following flowcharts provide example methods of embodiments of this disclosure. Regarding these example flowcharts, it should be noted that all blocks are not necessarily required to be performed. Moreover, additional blocks may be performed although they are not specifically illustrated in the example flowcharts. In addition, the example flowcharts are not mutually exclusive. For example, block(s) from one example flowchart may be performed in another of the example flowcharts.

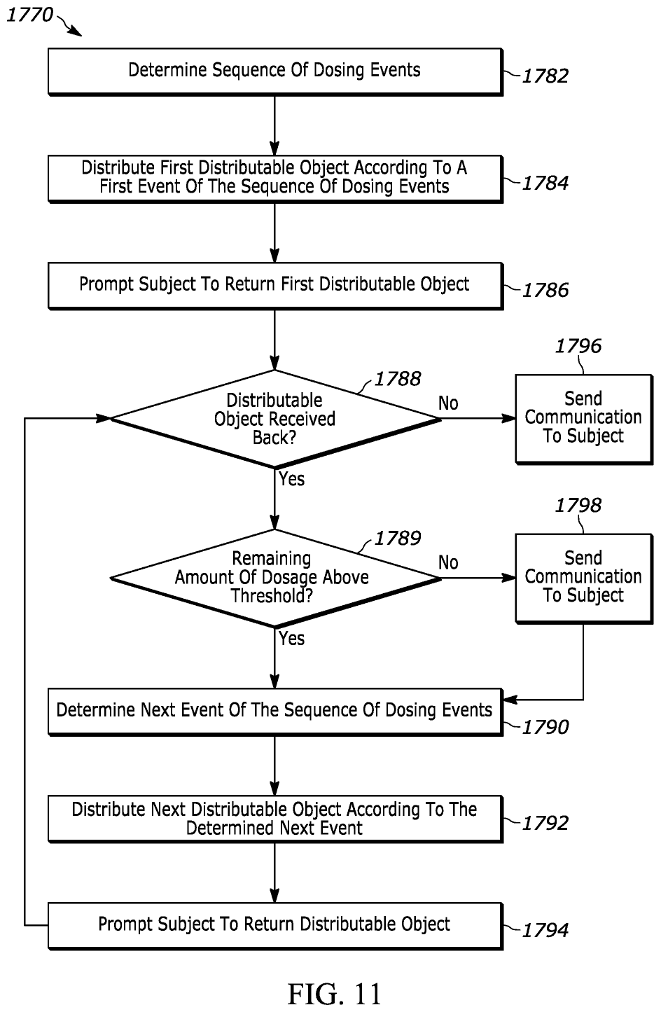

FIG. 11 illustrates an example flowchart 1770, including aspects of sequential dosing. The example method begins at block 1782 where the one or more processors 115 determine a sequence of dosing events. In some implementations, this is done by receiving the sequence of dosing events from the healthcare provider 106 (e.g., from a physician of the subject 172).

At block 1704, the one or more processors 115 control the distributor device 102 to distribute a first distributable object 170 to the subject 172. The first distributable object 170 may be distributed according to a first dosage event of the sequence of events. For example, the first distributable object 170 may be distributed at a date and time specified by the first dosage event, and with an amount of the ingestible ingredient specified by the first dosage event.

At block 1786, the one or more processors 115 control the distributor device 102 to prompt the subject 172 to return the first distributable object 170 to the distributor device 102. For instance, a visual prompt may be displayed at the display 126. Additionally or alternatively, a prompt may be displayed at a mobile device of the subject 172. Additionally or alternatively, the distributor device 102 may make an auditory announcement requesting that the subject 172 return the first distributable object 170.

At block 1788, the one or more processors 115 check if the distributable object 170 has been received back from the subject 172. If so, at block 1789, the one or more processors 115 determine if a remaining amount of the dosage on the returned distributable object is above a threshold. If not, at block 1798 the distributor device 102 may send a communication to the subject 172. For example, the communication may indicate that a next dosage may be modified due to the low amount of dosage on the returned distributable object. In another example, the communication may indicate that the medication will be stopped because the subject has ingested a higher dosage than the maximum assigned threshold of medication. The communication may be a visual communication displayed at display 126 or at a mobile device of the subject 172. Additionally or alternatively, the communication may be an auditory communication made by the distributor device 102.

If the answer at block at block 1789 is yes (or following the communication of block 1798, the one or more processors 115 determine a next dosage event of the sequence of dosing events at block 1790. In some examples, the next dosage event is determined by taking the next dosage event from the sequence of dosing events without modification. For instance, if no updates have been received from a physician, and the entire ingestible ingredient is gone from the distributable object 170, the next dosage event may be taken from the sequence of dosing events without modification. However, in some scenarios the next dosage event may be modified. For instance, if a physician has sent an update to increase or decrease the dosage, the next event may be modified accordingly. In another scenario, if the returned distributable object 170 still contains a predetermined amount of the consumable ingredient, the next dosage event may be modified (e.g., a dosage of the next dosage event may be modified).

At block 1792, the one or more processors 115 control the distributor device 102 to distribute the next distributable object 170 to the subject 172. The next distributable object 170 may be distributed according to the next dosage event determined at block 1790. For example, the next distributable object 170 may be distributed at a date and time specified by the next dosage event, and with an amount of the ingestible ingredient specified by the next dosage event.

At block 1794, the one or more processors 115 control the distributor device 102 to prompt the subject 172 to return the next distributable object 170 to the distributor device 102. For instance, a visual prompt may be displayed at the display 126. Additionally or alternatively, a prompt may be displayed at a mobile device of the subject 172. Additionally or alternatively, the distributor device 102 may make an auditory announcement requesting that the subject 172 return the first distributable object 170.

The example method then returns to block 1788, where the one or more processors 115 check if the distributable object 170 has been received back from the subject 172.

If the determination at block 1788 is no, the distributor device 102 sends a communication to the subject 172. For example, the communication may indicate that a next dosage will not be distributed until the previous distributable object 170 is received back. The communication may be a visual communication displayed at display 126 or at a mobile device of the subject 172. Additionally or alternatively, the communication may be an auditory communication made by the distributor device 102.

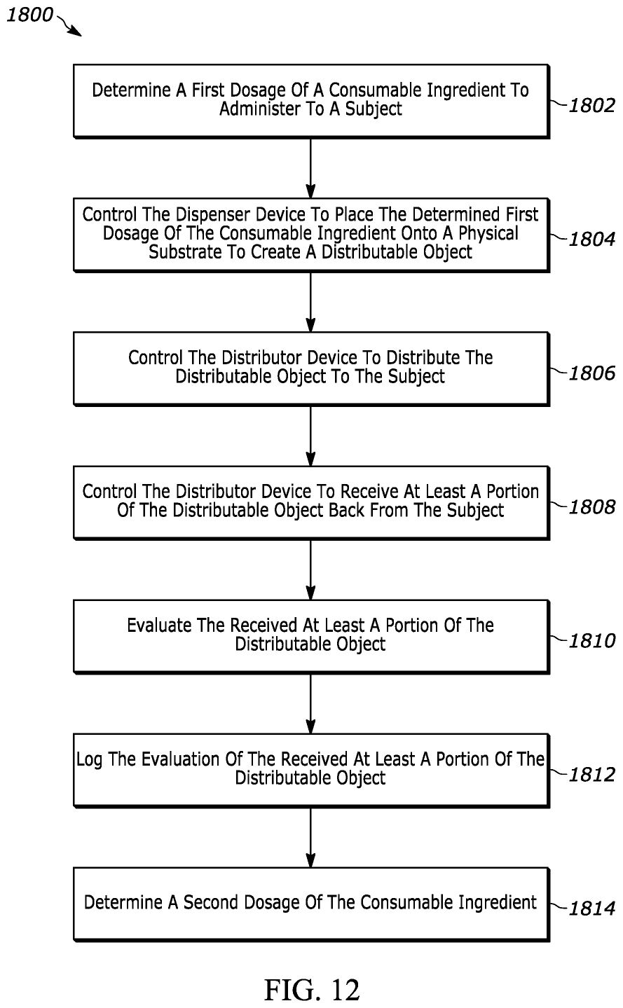

FIG. 12 illustrates an example flowchart 1800 for tracking patient medication protocol adherence. The blocks of the example flowchart 1800 may be performed by any suitable component or combination of components, such as the one or more processors 115 of the distributor device 102, the cloud computing platform 112, any of the patient devices, or any combination of these components, etc. However, for illustrative and exemplary purposes, the following discussion may refer to the one or more processors 115 as performing the blocks of the example flowchart 1800. Furthermore, it should be understood that the dispenser device, distributor device, physical substrate, distributable object, ingestible ingredient, etc. may refer to any such components/materials, as illustrated in any of the previous FIGS.

At block 1802, a first dosage of an ingestible ingredient to administer to a subject is determined (e.g., by the one or more processors 115). To this end, the one or more processors 115 may determine the subject by receiving login information of the subject (e.g., through the input device 128), such as a username and password. The subject may also be determined by receiving biometric data (e.g., fingerprint information, facial features from an image captured by a mobile device or by the camera 129 to be authenticated by a facial recognition technique, etc.) of the subject (e.g., through interface 128 and/or camera 129). In this regard, when (or as part of) determining the subject, the one or more processors 115 may determine a user profile (e.g., stored as part of the data 109a) of the subject (e.g., from the username, password, biometric data, etc. discussed above).

In other embodiments, a healthcare professional may enter information of multiple subjects. For example, a nurse may enter information of multiple subjects, and then subsequently pick up the distributable object(s) for delivery to each subject.

In one example, the determination of the first dosage may be made by the one or more processors 115 receiving the first dosage from an external system, such as the healthcare provider 106, the cloud computing platform 112, any of the patient devices, a physician system, a medicinal decisional support system, a hospital system, an assisted living facility system, etc.

At block 1804, the one or more processors 115 control the dispenser device to place the determined first dosage of the ingestible ingredient onto a physical substrate to create a distributable object. The dispenser device may include any kind or number of physical substrates. For example, the dispenser device may have physical substrates that are generally oval and flat (as in the example of FIGS. 2A and 2B). The determination of which physical substrate to use may be based on a subject preference (e.g., as included in the subject's user profile). Additionally or alternately, which physical substrate to use may be based on compatibility with a particular ingestible ingredient. For example, a particular API may not be compatible with a particular type of material that a physical substrate is made of.

The first dosage may be placed on the physical substrate by any suitable technique (e.g., any of the techniques described herein). In some examples, the dispenser device 160 may laminate, 3D print, spray coat, dip coat, vacuum coat, sputter, ink-jet print, dip-pen coat, etc. the ingestible ingredient onto the physical substrate. In another example, the dispenser device 160 may unspool the ingestible ingredient 162 from a section of tape in a cartridge to place the ingestible ingredient 160 onto the physical substrate 164. Moreover, any combination of techniques may be used.

Furthermore, in some example implementations block 1804 is not performed. For example, pre-coated distributable objects may be loaded into the dispenser device, so there is no need for the dispenser device to place the ingestible ingredient on a substrate.

In addition, at block 1804, as part of the creation of the distributable object, the system may place a quick response (QR) code on the distributable object. As will be described later below, the QR code may be useful in determining subsequent dosages of the ingestible ingredient to administer.

In some examples, the consumable ingredient may be an excipient, or an excipient in combination with another consumable ingredient substance, such as an API, as described above. In this regard, in some examples, the excipient is a flavor masking agent for altering a perceived taste of the ingestible ingredient (e.g., a medication) during provision of the ingestible ingredient to the subject orally. Thus, examples of the excipient include: methacrylic acid, methacrylic ester copolymers, aspartame, acesulfame potassium, sucralose, lemon flavor, menthol flavor, banana flavor, peppermint flavor, citric acid, sodium cyclamate, zinc sulfate. In some examples, the excipient is a delivery delay agent for delaying absorption of the ingestible ingredient to the subject after provision of the ingestible ingredient to the subject orally, including but not limited to polylactic acid (PLA), poly (lactic-co-glycolic acid) (PLGA), Polyethylene glycol (PEG), silk fibroin, their derivatives, and others. In some examples, the excipient is a tracking ingredient separate from the ingestible ingredient, such that an amount of the tracking ingredient associated with the distributable object after use by the subject is indicative of an amount of the ingestible ingredient used by the subject. In some examples, the tracking ingredient is barium fluoride and other radiopharmaceuticals, Fluorine-18, fluorescent dyes (ICG, IRDye8, Cy5.5, and many others as in 10.3389/fphar.2019.00510), lactulose, rhamnose, riboflavin, or MB-402/MB-301detectable in a bodily fluid of the subject. In some examples, the tracking ingredient is a metabolite of one or more compounds ingested, detectable by an external means, such as spectroscopy (examples are described extensively in https://doi.org/10.1038/s41746-019-0185-y).

Thus, more generally, an excipient may include flavor masking agent, a delaying agent, a texturing agent, a moisture responsive agent, a coloring agent, a temperature responsive agent, a delivery delay layer film, adhesion control layer, a protective layer, a tamper indicator, contamination indicator, pH sensing/indicating agent, and/or an enteric coating.

In some examples, the subject may be taking a placebo (e.g., the subject is part of a double-blind clinical study. In some such examples, the consumable ingredient may be the excipient alone.

At block 1806, the one or more processors 115 control the distributor device to distribute the distributable object to the subject. In some embodiments, this comprises opening a door, such as dispensary door 182, to allow a subject to take the distributable object (or to take a secured receptacle that the distributable object is in).

Moreover, in some embodiments, blocks 1804 and 1806 are performed simultaneously. For example, a spool of a cartridge may be rotated to place the first dosage on the substrate, while, simultaneously, a door of the distributor device is opened.

At block 1808, the one or more processors 115 control the distributor device to receive at least a portion of the distributable object back from the subject. For example, the distributable object may be received at an intake station, such as in the examples of FIGS. 3 and 4. In some embodiments, once received, the at least a portion of the distributable object is then sterilized, for example, as described elsewhere herein.

At block 1810, the received at least a portion of the distributable object may be evaluated. One purpose of evaluating the at least a portion of the distributable object may be to help in determining subsequent doses of the ingestible ingredient (e.g., a medication) to deliver to the subject. For instance, if the subject consumed only half of a medication (e.g., in the evaluation, half of the medication is determined to remain on the distributable object), a subsequent dose of the medication may be adjusted accordingly.

In some embodiments, the evaluation comprises determining a remaining amount of the ingestible ingredient on the at least a portion of the distributable object. The determination of the remaining portion may be done by any suitable technique. In one example, the at least a portion of the distributable object is weighed, and compared to a known weight of the distributable object. In another example, an imaging technique may be used. For instance, the at least a portion of the distributable object may be illuminated with light or UV light, and then an image or UV image may be captured of the at least a portion of the distributable object. Data from the image or UV image may then be used along with data from an imaging database or UV imaging database to determine a remaining portion of the ingestible ingredient. In this regard, a machine learning algorithm may also be used. For example, the data from the image or UV image may be input into a trained machine learning algorithm to determine the remaining portion of the ingestible ingredient.

In some embodiments, a QR code aids in the evaluation. For example, the camera 129 may capture an image of the subject placing the distributable object on her tongue. The captured image may include the QR code on the distributable object, which may be used to identify the ingestible ingredient that the subject is consuming. The QR code is an example indicia that when decoded results in decode data that may include any information, such as identification information of the ingestible ingredient, amount of the ingestible ingredient, information of the physical substrate of the distributable object, information of the subject, region code, etc. The identification of the ingestible ingredient via a QR code is particularly advantageous when the ingestible ingredient is a controlled substance because the identification provides further verification that the subject is consuming (rather than pocketing for resale) the controlled substance.

In one related example, a QR code or other indicia (e.g., a marking, a color, a decoding indicia) is placed on the substrate beneath the ingestible ingredient. Thus, when the subject consumes the ingestible ingredient, the QR code or other indicia may be used to provide identifying information, such as for identification of the ingestible ingredient, the substrate, the distributable object, etc.

At block 1812, the one or more processors 115 log the evaluation of the received at least a portion of the distributable object. For example, the one or more processors may record data of the evaluation in the distributor device itself (e.g., in the memory 120 and/or the database 118). Additionally or alternatively, the data of the evaluation may be recorded in an external database, such as that of the cloud computing platform 112, healthcare provider 106, and/or protocol database 108. The logging of such evaluation data advantageously helps compile data that may be used for patient care improvement, population health studies, clinical trials, etc.

In some embodiments, the logging of the evaluation data includes adding the evaluation to a subject's user profile (e.g., to the user profile information 109a of the protocol database 108). Advantageously, this allows building, over time, information of a patient's adherence to a protocol. For instance, over time, having this information in the user's profile allows the medication adherence protocol platform 140 to determine how often a subject misses a dose, what percentage of a dose the subject typically takes, etc. This may be useful for the medication adherence protocol platform 140 in determining future doses of the same or different ingestible ingredient for the subject. In another example, logging such information to the user profile may also be useful for the medication adherence protocol platform 140 in determining if the subject takes more of an ingestible ingredient if it is mixed with a particular taste additive.

In another example, logging the evaluation information in the user profiles may be useful in finding correlations between the ingestible ingredient and other features. For example, the cloud computing platform 112 may analyze user profiles of subjects that all recently underwent a particular operation. Because of the information added to the profiles from the evaluations, the cloud computing platform 112 may determine that the ingestible ingredient is typically being prescribed at too high or low a dosage.

At block 1814, the one or more processors 115 determine a second dosage of the ingestible ingredient. For example, the one or more processors 115 may have knowledge of a recurring dosage of a medication (e.g., the ingestible ingredient). If there is no ingestible ingredient or less than a predetermined amount of the ingestible ingredient on the returned at least a portion of the distributable object, the second dosage may simply be determined to be the recurring amount. On the other hand, if more than a predetermined amount of the ingestible ingredient is on the returned at least a portion of the distributable object, the recurring amount may be modified to determine the second dosage. Additionally or alternatively, the determination may be based on an update received from an external system (e.g., the healthcare provider 106, the cloud computing platform 112, etc.). For example, an update may indicate that the subject has also started taking an additional medication, and thus the recurring dosage of the first medication should be modified or even stopped. In another example, the update may indicate that the subject has stopped taking another medication. In another example, the update may indicate to stop the first medication (e.g., because of a determination by a physician). In yet another example, the update may indicate the amount of the second dosage (e.g., a physician determines a new dosage, and sends the update indicating the new dosage).

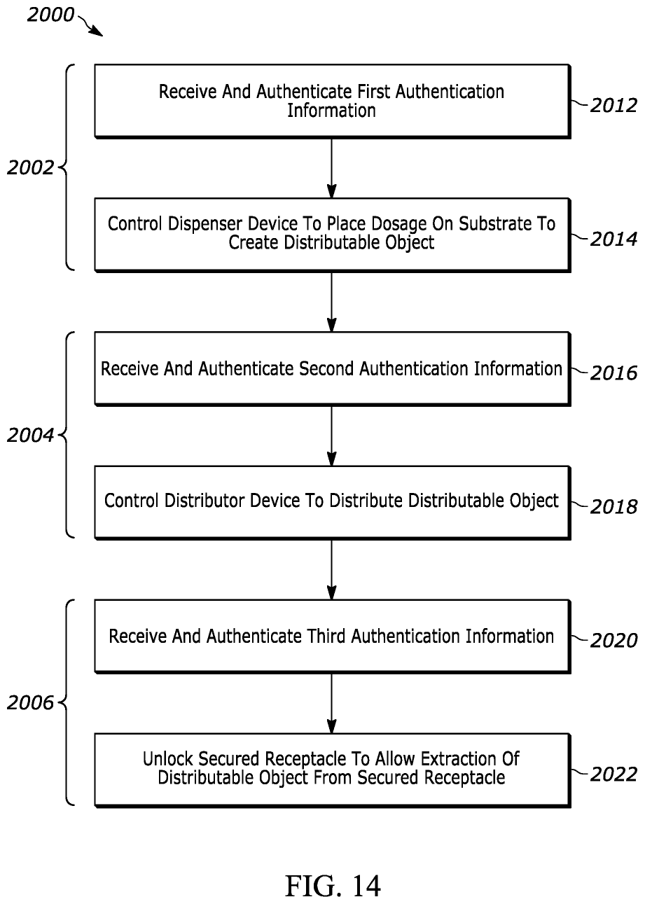

In some embodiments, a user profile (e.g., as determined at block 1802) of the subject is used to aid in the determination of the second dosage. The user profiles may be stored in any suitable location (e.g., at the distributor device 102, the could computing platform 112, and/or the healthcare provider 106). The user profiles may be retrieved (e.g., by the one or more processors 115) based on any suitable information. For example, the authentication information (e.g., discussed below with respect to FIG. 14) may be used to retrieve the user profile. In some embodiments, the user profile is retrieved in response to the subject entering a username and password (e.g., into the input device 128). In some embodiments, the user profile is retrieved in response to biometric data received from the subject.

The user profile may include any information about the user (e.g., the subject). Examples of such information include the subject's, name, gender, age, height, weight, date of birth, medical history, etc. Prescription and dosage information (e.g., of the ingestible ingredient) may also be included in the user profile.

The determination of the second dosage (e.g., at block 1814) may be further based on any of the information in the user profile. In some implementations, this includes determining a category that the subject belongs to. For example, the subject may belong to a particular category based on age, height, gender, and/or weight. Some categories may have predetermined dosage amounts for particular medications, so the one or more processors 115 may determine the second dosage amount to be the predetermined dosage amount for the category.

In another example, the user profile information (e.g., along with the data of the evaluation performed at block 1810) may be input into a trained machine learning algorithm to determine the second dosage amount.

Furthermore, data from the evaluation (e.g., performed at block 1810) may added to the user profile. This advantageously may improve future dose determinations of the ingestible ingredient for the subject.

In addition, the user profile may store medication information of other medications for the subject. In some examples, the one or more processors 115 determine a medication from the profile (e.g., the same medication of the ingestible ingredient for the first dosage, or a different medication altogether). If the medication is available at the distributor device, the distributor device may create and distribute a distributable object with the medication. If it is not available, the distributor device may determine a substitute medication available at the distributor device, and prompt the subject if the substitute medication is acceptable. If so, the distributor device creates and distributes a distributable object with the substitute medication.

In some implementations, based on the determined second dosage and/or information in the user profile, the one or more processors 115 may determine a predicted optimal time for a refill of a medication. The determination may be made by any suitable technique (e.g., look-up tables, statistical analysis, a machine learning algorithm, etc.).

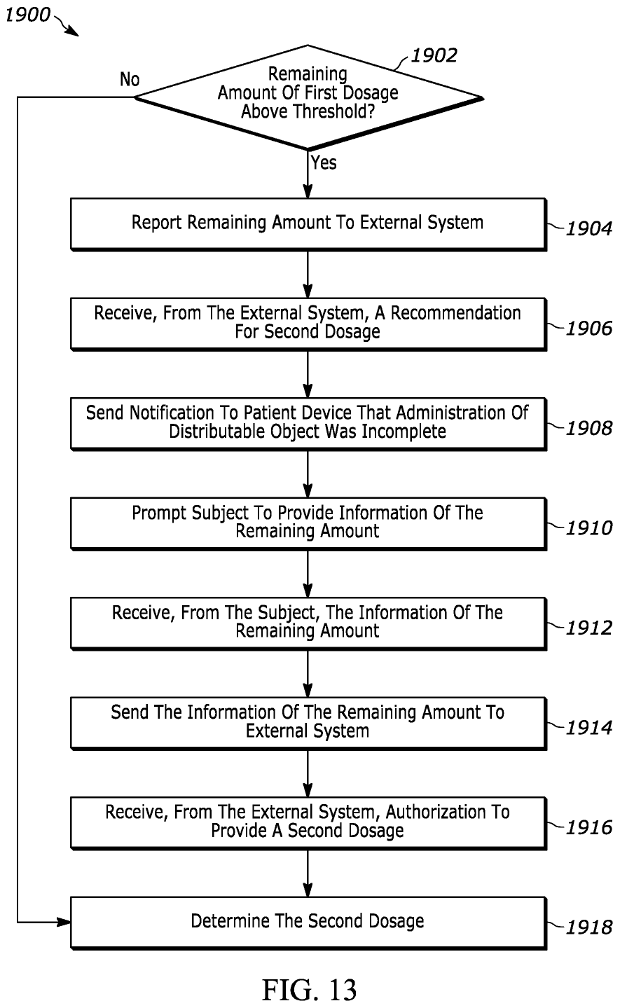

FIG. 13 shows an example flowchart 1900 illustrating exemplary actions that may be taken in response to a comparison between the remaining amount of the ingestible ingredient, and a predetermined threshold. As an initial matter, regarding the example flowchart 1900, it should be understood that the amount of the remaining first dosage may be done at any point in time, such as at block 1810 of FIG. 12.

At block 1902, the amount of the remaining dosage is compared to a predetermined threshold. In some embodiments, this comparison is simply a comparison between two weight values or two volume values (e.g., the threshold is a weight or volume threshold). In some embodiments, the comparison is a comparison based on a percentage remaining of the ingestible ingredient (e.g., the percentage of weight or volume remaining is compared to a percentage threshold).

If the remaining amount is above the threshold, the example method 1900 proceeds to blocks 1904-1908; however, it should be noted that, as mentioned above, not all blocks are required to be performed, and all blocks may be performed in any order.

At block 1904, the one or more processors 115 report the remaining amount to an external system (e.g., the healthcare provider 106, the cloud computing platform 112, etc.). The purpose of this is so that the external system may determine a second dosage for the patient. In determining the second dosage, the external system may use (in addition to the remaining amount) an information of the subject (e.g., medical records, etc.). Additionally or alternately, a healthcare provider (e.g., a physician) may simply view the remaining amount, and determine a second dosage for the subject.

At block 1904, the one or more processors 115 receive the recommendation from the external system.

Moving on, blocks 1908-1918 relate to actions that may have particular use with respect to controlled substances. For example, if a subject does not take the entire dosage of an addictive medication, this can sometimes be an indicator that the subject does not need the entire amount of a recurring dosage and so an amount of the recurring dosage should be reduced.

At block 1908, a notification is sent to a computing device of the subject (e.g., a patient device, such as the computing terminal 110A, the laptop computer 110B, the mobile cellular device 110C, and/or the mobile tablet computing device 110D). The notification may include any information. For example, the notification may indicate that administration of the distributable object and/or ingestible ingredient was incomplete. In another example, the notification may indicate the remaining amount (e.g., as a numeric volume or weight; or as a percentage volume or weight). In another example, the notification may indicate a time that the next dose will be available, or indicate that a physician has been contacted regarding the remaining amount of the first dosage.

At block 1910, the subject may be prompted (e.g., at the computing device of the subject or at the display 126) to provide information regarding the remaining amount. For instance, the patient may be prompted to input information as to why half of the first dose was returned. The subject may also be prompted to indicate how much of the ingestible ingredient the subject has (e.g., if the subject has consumed the ingestible ingredient on other distributable objects that the subject may have), as this information may be useful in tracking or restricting controlled substances.

At block 1912, the one or more processors 115 may receive information of the remaining amount from the subject. For instance, the subject may indicate that he consumed only half of the dose because the medication was a painkiller and only half of the dose was needed to effectively alleviate the pain. The received information may also include information about amounts of the ingestible ingredient the subject still has in his possession.

At block 1914, the one or more processors 115 sends the information received from the subject to an external system (e.g., the healthcare provider 106, the cloud computing platform 112, etc.). The external system may then, based on the received information, authorize providing a second dosage to the subject. For example, a physician or computer algorithm at the external system may make the determination of whether or not to authorize the second dosage. For instance, a physician or computer algorithm at the external system may make the determination based on factors, such as if the ingestible ingredient is a controlled substance, remaining amount of the ingestible ingredient, subject information (e.g., any information in the user profile, including criminal history of the subject), the information of the remaining amount provided by the subject, etc. If a computer algorithm is used, such factors may be input into a lookup table or machine learning algorithm to determine if authorization should be granted. Additionally or alternatively, a physician may review all of the relevant information to determine if authorization should be granted.

In a variation on this, the external system may determine the second dosage to administer as well as determining if authorization should be granted.

At block 1916, the one or more processors 115 receives, from the external system the authorization for a second dosage, and/or determined amount of the second dosage.

At block 1920, the one or more processors 115, after receiving the authorization from the external system, determines the amount of the second dosage to administer to the subject (e.g., proceeds as in block 1814 of the example of FIG. 12.). In some variations where the external system made the determination of the second dosage, the one or more processors simply sets the second dosage to be the amount determined by the external system.

Returning now to block 1902, if the remaining amount of the first dosage is not above the threshold, the one or more processors simply proceeds to block 1918 determine the second dosage.

FIG. 14 illustrates an example flowchart 2000 for actions relating to authentication and authorization. Broadly speaking, in some embodiments, the example flowchart 2000 comprises three phases: a first phase 2002 for authorization to create a distributable object; a second phase 2004 to distribute the distributable object; and a third phase 2006 to extract the distributable object from a secured receptacle.

As an initial matter, it should be understood that any of the “authentication information” (e.g., the first, second, or third authentication information, or any other authentication information) may comprise authentication information of any kind. For example, the authentication information may be a passcode and/or password (e.g., entered via the input device 128, or the I/O components 1750, etc.). In another example, the authentication information comprises biometric information of a subject (e.g., fingerprint information, facial features from an image captured by a mobile device or by the camera 129 to be authenticated by a facial recognition technique, etc.). In yet another example, the authentication information comprises a QR code displayed on, for example, a mobile device of the subject, and captured by the camera 129.

Returning to the example flowchart 2000, at block 2012, the one or more processors 115, receive and authenticate first authentication information. The first authentication information may, for example, be authentication information as described above (e.g., a passcode, password, biometric data, etc.). The first authentication information may also be received, for example, as described above (e.g., received via the user input device 128, etc.). To authenticate the first authentication information, the received authentication information may be compared to information stored locally at the distributor device 102 and/or at the cloud computing platform 112.

At block 2014, based on the successful first authentication, the one or more processors 115 may control the dispenser device to place the ingestible ingredient on the physical substrate to create the distributable object (e.g., perform block 1804 of the example of FIG. 12).

At block 2016, the example method 2000 enters the second phase 2004 (e.g., authorization to distribute the distributable object). Here, the one or more processors 115, receive and authenticate second authentication information. The second authentication information may, for example, be authentication information as described above (e.g., a passcode, password, biometric data, etc.). The second authentication information may also be received, for example, as described above (e.g., received via the user input device 128, etc.). To authenticate the second authentication information, the received second authentication information may be compared to information stored locally at the distributor device 102 and/or at the cloud computing platform 112.

At block 2018, based on the successful second authentication, the one or more processors 115 may control the distributor device to distribute the distributable object to the subject (e.g., a patient, a nurse, a medical provider, a caregiver, etc.). In some implementations, this comprises performing block 1806 of the example of FIG. 12. In some embodiments, the distributable object is distributed in a secured receptacle to the subject, whereas, in other embodiments, the distributed object is distributed directly to the subject without a secured receptacle.

At block 2020, the example method 2000 enters the third phase (e.g., opening the secured receptacle). Here, the one or more processors of the secured receptacle 1720, may receive and authenticate third authentication information.

The third authentication information may, for example, be authentication information as described above (e.g., a passcode, password, biometric data, etc.). The third authentication information may also be received, for example, as described above (e.g., received via the user input device 128, etc.). To authenticate the second authentication information, the received third authentication information may be compared to information stored at the distributor device 102, at the cloud computing platform 112, and/or at the secured receptacle. In some embodiments, if the authorization is done at the one or more processors 115, or the cloud computing platform 112, the authorization may be sent to the one or more processors of the secured receptacle 1720.

At block 2022, based on the successful third authentication, the one or more processors of the secured receptacle 1720 may authorize extraction of the distributable object from the secured receptacle. For example, the one or more processors of the secured receptacle 1720 may unlock the lockable opening 1710.

In some implementations (e.g., when the distributor device is located in the home of a patient), each of the first, second, and third authentication information may come from the same subject (e.g., the patient). In such implementations, it may be optimal to combine/collapse phase 2004 and phase 2006, or even combine/collapse all of phases 2002, 2004 and 2006 to avoid duplicative authentication. However, in other implementations, it is advantageous to perform each phase separately. One such example is a hospital setting where a nurse collects multiple secured receptacles for multiple patients from the distributor device. In one implementation of this example, the nurse may provide the first and second authentication information so that the distributor device creates the distributable objects and distributes them in secured receptacles to the nurse. The nurse may then deliver the secured receptacles to each patient, and each patient may individually provide the third authentication information to open their secured receptacle.

FIGS. 15B and 15C illustrate additional examples of closed loop systems for implementing the processes described herein. The system 2130 in FIG. 15B uses a distributor device 2134 to dispense a distributable object 2138 to a subject 2132 and uses a separate receiver device 2136 to receive and analyze the distributable object 2138′ after use by the subject 2132. The system 2150 in FIG. 15C is similar to the system 2130, but a single distributor device 2154 is able to dispense distribute object 2158 to a subject 2152, receive the distributable object 2152′ after use, and analyze the same.

In an example operation of the example closed loop systems 2130, 2150, the distributor devices 2134, 2154 may receive a prescription of a prescribed dosage of an ingestible ingredient, for example a prescription received via network (not shown) from an external computing system, such as a physician's computing system, pharmacy, insurance provider, etc. Then, the subjects 2132, 2152 are authenticated as an authorized user of the distributor device 2134, 2136, and 2154, respectively. In some examples, authentication is of a patient and/or a physician and/or a care provider. Once authentication is achieved, the distributor device 2134, 2154 then produces the distributable objects 2138, 2158 respectively, each containing a respective dose of an ingestible ingredient 2140, 2160, and provides them to the respective subjects 2132, 2152. The subjects 2132, 2152 administer the ingestible ingredient, ideally as prescribed. After administration, the distributable objects 2138′, 2158′, respectively, are deposited back into a distributor device (distributor device 2136 in the example of FIG. 15B, and distributor device 2154 in the example of FIG. 15C), which logs (and optionally analyzes) the used distributable objects 2138, 2158, respectively. Relevant information about the distributable objects 2138, 2158 (and optionally about the subjects 2132, 2152) is fed back (optionally via a network) to the distributor device 2134, 2154. In this manner, control over the dispensing of the follow-on dose via a distributable object 2138, 2158 is enabled. Furthermore, information collected throughout the process may be used to determine the composition of the next distributable object 2138, 2158.

FIGS. 15A and 15B illustrate examples including multiple subjects 2132, 2152. However, it should be understood that these examples apply as well when only a single subject is involved.

### Additional Exemplary Embodiments

Aspect 1. A method for tracking consumption of an ingestible ingredient, the method comprising:
determining, via one or more processors, a first dosage of an ingestible ingredient to administer to a subject;
placing the determined first dosage of the ingestible ingredient onto a physical substrate to create a distributable object;
distributing the distributable object to the subject;
receiving at least a portion of the distributable object back from the subject;
evaluating, via the one or more processors, the received at least a portion of the distributable object; and
logging, via the one or more processors, the evaluation of the received at least a portion of the distributable object.

Aspect 2. The method of aspect 1, based on the evaluation, via the one or more processors, determining a second dosage of the ingestible ingredient.

Aspect 3. The method of any one of aspects 1-2, wherein the evaluating the at least a portion of the distributable object comprises determining a remaining amount of the first dosage.

Aspect 4. The method any one of aspects 1-3, further comprising determining a second dosage amount by changing a recurring dosage amount based on the determined remaining amount of the first dosage.

Aspect 5. The method of any one of aspects 1-4, wherein the evaluating the at least a portion of the distributable object comprises determining a remaining amount of the first dosage, and the method further comprises:
if the remaining amount of the first dosage is above a predetermined threshold, reporting, via the one or more processors, the remaining amount of the first dosage to an external system; and
receiving, in response to the reporting of the first dosage to the external system, via the one or more processors, a recommendation for a second dosage of the ingestible ingredient from the external system.

Aspect 6. The method of any one of aspects 1-5, wherein the external system is a physician system, a medicinal decisional support system, a hospital system, or an assisted living facility system.

Aspect 7. The method of any one of aspects 1-6, wherein the evaluating the at least a portion of the distributable object comprises determining a remaining amount of the first dosage, and the method further comprises:
if the remaining amount of the first dosage is above a predetermined threshold, sending, via the one or more processors, to a computing device of the subject, a notification indicating that administration of the distributable object was incomplete.

Aspect 8. The method of any one of aspects 1-7, wherein the evaluating the at least a portion of the distributable object comprises determining a remaining amount of the first dosage, and the method further comprises:
if the remaining amount of the first dosage is above a predetermined threshold, prompting, via the one or more processors, the subject to provide information of the remaining amount;
providing, via the one or more processors, the information of the remaining amount to an external system;
receiving, via the one or more processors and from the external system, in response to the provision of the information of the remaining amount, authorization to provide a second dosage of the ingestible ingredient to the subject; and
in response to receiving the authorization, distributing the second dosage of the ingestible ingredient to the subject.

Aspect 9. The method of any one of aspects 1-8, wherein the physical substrate is a first physical substrate, and the method further comprises:
in response to receiving the authorization, and prior to distributing the second dosage of the ingestible ingredient, placing the second dosage of the ingestible ingredient on either the first physical substrate or a second physical substrate.

Aspect 10a. The method of any one of aspects 1-9, further comprising:
in response to receiving the authorization, and prior to distributing the second dosage of the ingestible ingredient, selecting a pre-coated distributable object with an amount of the ingestible ingredient within a predetermined tolerance range of the second dosage.

Aspect 10b. The method of any one of aspects 1-10a, wherein the evaluating the at least a portion of the distributable object comprises:
illuminating the at least a portion of the distributable object with ultraviolet (UV) light; and
capturing an UV image of the at least a portion of the distributable object based on the illumination.

Aspect 10c. The method of any one of aspects 1-10b, wherein the evaluating the at least a portion of the distributable object comprises:
illuminating the at least a portion of the distributable object with ultraviolet (UV) light;
capturing an UV image of the at least a portion of the distributable object based on the illumination; and
comparing data of the UV image to data from an UV imaging database, wherein the data from the UV imaging database comprises: (i) UV image data, and (ii) ingestible ingredient data mapped to the UV image data.

Aspect 10d. The method of any one of aspects 1-10c, wherein the evaluating the at least a portion of the distributable object comprises:
weighing the at least a portion of the distributable object to determine a weight of the distributable object; and
determining a remaining amount of the first dosage based on the weight.

Aspect 10e. The method of any one of aspects 1-10d, wherein the evaluating the at least a portion of the distributable object comprises:
capturing a visual image of the at least a portion of the distributable object; and
inputting data of the visual image into a machine learning algorithm to determine a remaining amount of the first dosage.

Aspect 10f. The method of any one of aspects 1-10e, further comprising:
receiving, via the one or more processors, from an external system, an update; and
based on the evaluation and the received update, via the one or more processors, determining a second dosage of the ingestible ingredient.

Aspect 11. The method of any one of aspects 1-10f, further comprising:
identifying, via the one or more processors, a user profile of the subject including subject information of the user, wherein the subject information includes: an identity of the subject, an age of the subject, a gender of the subject, a weight of the subject, and/or a height of the subject; and
determining, via the one or more processors, a second dosage of the ingestible ingredient based on: (i) the subject information, and (ii) an amount of the ingestible ingredient detected during the evaluation.

Aspect 12. The method of any one of aspects 1-11, further comprising:
identifying, via the one or more processors, a user profile of the subject including subject information of the user, wherein the subject information includes: an age of the subject, a gender of the subject, a weight of the subject, and/or a height of the subject; and
determining, via the one or more processors, a second dosage of the ingestible ingredient by:
determining, based on the subject information, that the subject belongs to a category of users that has a predetermined dosage amount of the ingestible ingredient; and
determining the second dosage to be the predetermined dosage amount.

Aspect 13. The method of any one of aspects 1-12, further comprising:
identifying, via the one or more processors, a user profile of the subject; and
based on the evaluation, via the one or more processors, determining a second dosage of the ingestible ingredient by inputting, into a trained machine learning algorithm: (i) data of the evaluation of the received at least a portion of the distributable object, and (ii) subject information of the identified user profile of the subject.

Aspect 14a. The method of any one of aspects 1-13, further comprising:
identifying, via the one or more processors, a user profile of the subject; and adding, via the one or more processors, data of the evaluated at least a portion of the distributable object to the user profile.

Aspect 14b. The method of any one of aspects 1-14a, wherein the placing the determined first dosage of the ingestible ingredient onto the physical substrate to create the distributable object comprises:
laminating the determined first dosage of the ingestible ingredient onto the physical substrate;
three-dimensional (3D) printing the determined first dosage of the ingestible ingredient onto the physical substrate;
spray coating the determined first dosage of the ingestible ingredient onto the physical substrate;
dip coating the determined first dosage of the ingestible ingredient onto the physical substrate; or
unspooling, from a cartridge, a section of a tape coated with the ingestible ingredient onto the physical substrate.

Aspect 14b. The method of any one of aspects 1-14a, further comprising, subsequent to the placing the determined first dosage of the ingestible ingredient onto the physical substrate, placing an excipient onto the ingestible ingredient.

Aspect 14c. The method of any one of aspects 1-14b, further comprising, subsequent to the placing of the determined first dosage of the ingestible ingredient onto the physical substrate, affixing a quick response (QR) code onto the distributable object.

Aspect 14d. The method of any one of aspects 1-14c, further comprising:
subsequent to the placing of the determined first dosage of the ingestible ingredient onto the physical substrate, affixing a quick response (QR) code onto the distributable object; and
based on the evaluation, via the one or more processors, determining a second dosage of the ingestible ingredient; and
wherein the determining of the second dosage is further based on evaluating a received image including: (i) the QR code, and (ii) the subject's tongue.

Aspect 14e. The method of any one of aspects 1-14d, further comprising, subsequent to the receiving at least a portion of the distributable object back from the subject, sterilizing the received at least a portion of the distributable object.

Aspect 15. The method of any one of aspects 1-14e, further comprising:
receiving, via the one or more processors, biometric data from the subject; and attempting, via the one or more processors, to authenticate the subject based on the biometric data; and
wherein the distributing the distributable object to the subject occurs in response to a successful authentication of the subject.

Aspect 16. The method of any one of aspects 1-15, further comprising:
receiving, via the one or more processors, biometric data from the subject;
identifying, via the one or more processors, a user profile of the subject based on the biometric data;
determining, via the one or more processors, if a medication from the user profile is available;
if the determined medication is not available, determining, via the one or more processors, if a substitute ingestible ingredient for the medication is available; and
if the substitute ingestible ingredient is available, prompting, via the one or more processors, the subject to accept or decline a substitute medication corresponding to the substitute ingestible ingredient.

Aspect 17. The method of any one of aspects 1-16, wherein the distributing the distributable object to the subject comprises distributing the distributable object in a secured receptacle to the subject.

Aspect 18. The method of any one of aspects 1-17, further comprising:
authenticating, via the one or more processors, first authorization information, wherein the placing of the determined first dosage of the ingestible ingredient onto the physical substrate occurs in response to the authentication of the first authorization information;
authenticating, via the one or more processors, second authentication information, wherein the distributing the distributable object: (i) occurs in response to the authentication of the second authentication information, and (ii) comprises distributing the distributable object into a secured receptacle;
authenticating, via one or more processors of the secured receptacle, third authentication information; and
in response to the authentication of the third authentication information, unlocking the secured receptacle to thereby allow extraction of the distributable object.

Aspect 19. The method of any one of aspects 1-18, further comprising:
authenticating, via the one or more processors, first authorization information, wherein the placing of the determined first dosage of the ingestible ingredient onto the physical substrate occurs in response to the authentication of the first authorization information; and
authenticating, via the one or more processors, second authentication information, wherein the distributing the distributable object occurs in response to the authentication of the second authentication information.

Aspect 20. The method of any one of aspects 1-19, further comprising:
authenticating, via the one or more processors, authorization information, wherein the placing of the determined first dosage of the ingestible ingredient onto the physical substrate occurs in response to the authentication of the authorization information; and
wherein the authentication information comprises biometric data, passcode data, and/or data from a mobile device of the subject.

Aspect 21. The method of any one of aspects 1-20, further comprising:
identifying, via the one or more processors, a user profile of the subject; determining, via the one or more processors, if a replenishment of a medication from the user profile is available; and
if the replenishment of the medication is available, distributing a second distributable object comprising the replenishment of the medication to the subject.

Aspect 22. The method of any one of aspects 1-21, further comprising:
based on the evaluation, via the one or more processors, determining a second dosage of the ingestible ingredient; and
based on the determined second dosage, via the one or more processors, calculating a predicted optimal time for a refill of the ingestible ingredient.

Aspect 23. The method of any one of aspects 1-22, wherein the determining of the first dosage comprises receiving, via the one or more processors, the first dosage amount from an external database.

Aspect 24. The method of any one of aspects 1-23, wherein the determining of the first dosage comprises receiving, via the one or more processors, the first dosage amount from a computing device of the subject.

Aspect 25. The method of any one of aspects 1-24, further comprising:
determining, via the one or more processors, a sequence of dosing events, wherein the first dosage corresponds to a first event of the sequence of dosing events; and
sequentially distributing subsequent distributable objects to the subject, wherein each distribution event of the sequential distribution: (i) occurs in response to receiving authorization based on receiving a previously distributed distributable object back from the subject, and (ii) is logged via the one or more processors.

Aspect 26. The method of any one of aspects 1-25, further comprising loading a cartridge containing the ingestible ingredient into a medication dispenser apparatus, wherein the cartridge is configured such that, upon tampering, heat is released to deactivate the ingestible ingredient.

Aspect 27. The method of any one of aspects 1-26, further comprising loading a cartridge containing the ingestible ingredient into a medication dispenser apparatus, wherein the cartridge is configured such that, upon tampering, heat is released to deactivate the ingestible ingredient, and wherein the heat is released by:
releasing the heat from a thermal battery of a spool of the cartridge; or
releasing the heat from a resistively heated element of the spool of the cartridge.

Aspect 28. The method of any one of aspects 1-27, further comprising loading a cartridge containing the ingestible ingredient into a medication dispenser apparatus, wherein the cartridge is configured such that, upon tampering, gas is released to deactivate the ingestible ingredient.

Aspect 29. A system for tracking consumption of an ingestible ingredient, the system comprising:
a distributor device comprising a housing, and a communication interface;
a dispenser device housed within the distributor device, the dispenser device being configured to place dosages of ingestible ingredients onto physical substrates; and
one or more processors configured to:
   determine a first dosage of an ingestible ingredient to administer to a subject;
   control the dispenser device to place the determined first dosage of the ingestible ingredient onto a physical substrate to create a distributable object;
   control the distributor device to distribute the distributable object to the subject;
   control the distributor device to receive at least a portion of the distributable object back from the subject;
   evaluate the received at least a portion of the distributable object; and
   log the evaluation of the received at least a portion of the distributable object.

Aspect 30. The system of aspect 39, further comprising a cartridge comprising a spool and a locking mechanism; and
wherein the locking mechanism is configured to:
   prohibit the spool from rotating when the locking mechanism is in a locked position;
   allow the spool to rotate when the locking mechanism is not in the locked position; and
   change between the locked position and the unlocked position based on a received signal.

Aspect 31. The system of any one of aspects 29-30, further comprising a cartridge comprising one or more cartridge processors configured to communicate:
a location of the cartridge;
a status of the cartridge;
an access history of the cartridge;
a temperature of the cartridge; or
identifying information of the cartridge.

Aspect 32. The system of any one of aspects 29-31, further comprising a cartridge comprising: (i) a spool, and (i) a tape including the ingestible ingredient and a waxed paper backing.

Aspect 33. The system of any one of aspects 29-32, further comprising a cartridge housing physical substrates with no ingestible ingredient thereon.

Aspect 34. The system of any one of aspects 29-33, further comprising a cartridge housing physical substrates with an ingestible ingredient affixed thereon.

Aspect 35. The system of any one of aspects 29-34, wherein the one or more processors are configured to control the distributor device to distribute the distributable object to the subject by opening a door of the distributor device.

Aspect 36. The system of any one of aspects 29-35, wherein the one or more processors are configured to simultaneously:
(i) rotate a spool of a cartridge to place the determined first dosage of the ingestible ingredient onto the physical substrate to create the distributable object; and
(ii) open a door of the distributor device to distribute the distributable object to the subject.

Aspect 37. The system of any one of aspects 29-36, wherein dispenser device is further configured to allow a user to replace a first cartage with a second cartridge, and wherein the second cartridge comprises the ingestible ingredient.

Aspect 38. The system of any one of aspects 29-37, wherein the dispenser device is further configured to, if an amount of the ingestible ingredient in the dispenser device falls below a predetermined level:
provide an alert indicating that the amount of ingestible ingredient in the dispenser device is low; and
unlock a first cartridge from the dispenser device, thereby allowing the first cartridge to be removed and replaced with a second cartridge.

Aspect 39. The system of any one of aspects 29-38, wherein the one or more processors are further configured to send data of the evaluation to a database.

Aspect 40. The system of any one of aspects 29-39, wherein:
the one or more processors are further configured to open a door of the distributor device to allow the at least a portion of the distributable object to be deposited in the dispenser device;
the dispenser device further comprises a camera configured to capture a visual image of the at least a portion of the distributable object; and
the one or more processors are further configured to input data of the visual image into a machine learning algorithm to determine a remaining amount of the first dosage.

Aspect 41. The system of any one of aspects 29-40, wherein:
the one or more processors are further configured to open a door of the distributor device to allow the at least a portion of the distributable object to be deposited in the dispenser device;
the dispenser device further comprises an ultraviolet (UV) camera configured to capture an UV image of the at least a portion of the distributable object; and the one or more processors are further configured to input data of the UV image into a machine learning algorithm to determine a remaining amount of the first dosage.

Aspect 42. The system of any one of aspects 29-41, wherein:
the one or more processors are further configured to open a door of the distributor device to allow the at least a portion of the distributable object to be deposited in the dispenser device;
the dispenser device further comprises a scale configured to weigh the at least a portion of the distributable object; and
the one or more processors are further configured to evaluate the received at least a portion of the distributable object by determining a remaining amount of the ingestible ingredient based on weight information received from the scale.

Aspect 43. A system for tracking consumption of an ingestible ingredient, the system comprising:
a plurality of warehouses;
a plurality of vehicles;
a distributor device comprising a housing, and a communication interface;
a dispenser device housed within the distributor device, the dispenser device being configured to place dosages of the ingestible ingredients onto physical substrates; and
one or more processors configured to:
   determine a first dosage of an ingestible ingredient to administer to a subject;
   control the dispenser device to place the determined first dosage of the ingestible ingredient onto a physical substrate to create a distributable object;
   control the distributor device to distribute the distributable object to the subject;
   control the distributor device to receive at least a portion of the distributable object back from the subject;
   evaluate the received at least a portion of the distributable object; and
   log the evaluation of the received at least a portion of the distributable object.

Aspect 44. A method for tracking consumption of an ingestible ingredient, the method comprising:
determining, via one or more processors, a sequence of dosing events;
distributing a first distributable object to a subject, the first distributable object including a first dosage of an ingestible ingredient specified by a first dosing event of the sequence of dosing events; and
sequentially distributing subsequent distributable objects to the subject, wherein each distribution event of the sequential distribution: (i) occurs in response to receiving authorization based on receiving a previously distributed distributable object back from the subject, and (ii) is logged via the one or more processors.

Aspect 45. The method of aspect 44, further comprising receiving, via the one or more processors, an updated sequence of dosing events from an external system, the external system being a physician system, a medicinal decisional support system, a hospital system, or an assisted living facility system; and
wherein distribution events of the sequential distribution are done according to the updated sequence of dosing events.

Aspect 46. The method of any one of aspects 44-45, further comprising:
identifying, via the one or more processors, a user profile of the subject including subject information of the user, wherein the subject information includes: an age of the subject, a gender of the subject, a weight of the subject, and/or a height of the subject; and
wherein the sequential distribution further includes logging each distribution event to the user profile.

Aspect 47. The method of any one of aspects 44-46, further comprising:
receiving used distributable objects back from the subject;
evaluating the received used distributable objects; and
updating the sequence of dosing events based on the evaluations of the used distributable object; and
wherein the sequential distribution of the subsequent distributable objects is done according to the updated sequence of dosing events.

Aspect 48. The method of any one of aspects 44-47, wherein the distribution events of the sequential distribution are logged to a distributed ledger.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A method for tracking consumption of an ingestible ingredient, the method comprising:
determining, via one or more processors, a first dosage of an ingestible ingredient to administer to a subject;
placing the determined first dosage of the ingestible ingredient onto a physical substrate to create a distributable object;
distributing the distributable object to the subject;
receiving at least a portion of the distributable object back from the subject;
evaluating, via the one or more processors, the received at least a portion of the distributable object; and
logging, via the one or more processors, the evaluation of the received at least a portion of the distributable object.

2. The method of claim 1, based on the evaluation, via the one or more processors, determining a second dosage of the ingestible ingredient.

3. The method of claim 1, wherein the evaluating the at least a portion of the distributable object comprises determining a remaining amount of the first dosage.

4. The method of claim 3, further comprising determining a second dosage amount by changing a recurring dosage amount based on the determined remaining amount of the first dosage.

5. The method of claim 1, wherein the evaluating the at least a portion of the distributable object comprises determining a remaining amount of the first dosage, and the method further comprises:
if the remaining amount of the first dosage is above a predetermined threshold, reporting, via the one or more processors, the remaining amount of the first dosage to an external system; and
receiving, in response to the reporting of the first dosage to the external system, via the one or more processors, a recommendation for a second dosage of the ingestible ingredient from the external system.

6. The method of claim 5, wherein the external system is a physician system, a medicinal decisional support system, a hospital system, or an assisted living facility system.

7. The method of claim 1, wherein the evaluating the at least a portion of the distributable object comprises determining a remaining amount of the first dosage, and the method further comprises:
if the remaining amount of the first dosage is above a predetermined threshold, sending, via the one or more processors, to a computing device of the subject, a notification indicating that administration of the distributable object was incomplete.

8. The method of claim 1, wherein the evaluating the at least a portion of the distributable object comprises determining a remaining amount of the first dosage, and the method further comprises:
if the remaining amount of the first dosage is above a predetermined threshold, prompting, via the one or more processors, the subject to provide information of the remaining amount;
providing, via the one or more processors, the information of the remaining amount to an external system;
receiving, via the one or more processors and from the external system, in response to the provision of the information of the remaining amount, authorization to provide a second dosage of the ingestible ingredient to the subject; and
in response to receiving the authorization, distributing the second dosage of the ingestible ingredient to the subject.

9. The method of claim 8, wherein the physical substrate is a first physical substrate, and the method further comprises:
in response to receiving the authorization, and prior to distributing the second dosage of the ingestible ingredient, placing the second dosage of the ingestible ingredient on either the first physical substrate or a second physical substrate.

10. The method of claim 8, further comprising:
in response to receiving the authorization, and prior to distributing the second dosage of the ingestible ingredient, selecting a pre-coated distributable object with an amount of the ingestible ingredient within a predetermined tolerance range of the second dosage.

11. The method of claim 1, further comprising:
identifying, via the one or more processors, a user profile of the subject including subject information of the user, wherein the subject information includes: an identity of the subject, an age of the subject, a gender of the subject, a weight of the subject, and/or a height of the subject; and
determining, via the one or more processors, a second dosage of the ingestible ingredient based on: (i) the subject information, and (ii) an amount of the ingestible ingredient detected during the evaluation.

12. The method of claim 1, further comprising:
identifying, via the one or more processors, a user profile of the subject including subject information of the user, wherein the subject information includes: an age of the subject, a gender of the subject, a weight of the subject, and/or a height of the subject; and
determining, via the one or more processors, a second dosage of the ingestible ingredient by:
determining, based on the subject information, that the subject belongs to a category of users that has a predetermined dosage amount of the ingestible ingredient; and
determining the second dosage based on: (i) the predetermined dosage amount, and (ii) data of the evaluation of the received at least a portion of the distributable object.

13. The method of claim 1, further comprising:
identifying, via the one or more processors, a user profile of the subject; and
based on the evaluation, via the one or more processors, determining a second dosage of the ingestible ingredient by inputting, into a trained machine learning algorithm: (i) data of the evaluation of the received at least a portion of the distributable object, and (ii) subject information of the identified user profile of the subject.

14. The method of claim 1, further comprising:
identifying, via the one or more processors, a user profile of the subject; and
adding, via the one or more processors, data of the evaluated at least a portion of the distributable object to the user profile.

15. The method of claim 1, further comprising:
receiving, via the one or more processors, biometric data from the subject; and
attempting, via the one or more processors, to authenticate the subject based on the biometric data; and
wherein the distributing the distributable object to the subject occurs in response to a successful authentication of the subject.

16. The method of claim 1, further comprising:
receiving, via the one or more processors, biometric data from the subject;
identifying, via the one or more processors, a user profile of the subject based on the biometric data;
determining, via the one or more processors, if a medication from the user profile is available;
if the determined medication is not available, determining, via the one or more processors, if a substitute ingestible ingredient for the medication is available; and
if the substitute ingestible ingredient is available, prompting, via the one or more processors, the subject to accept or decline a substitute medication corresponding to the substitute ingestible ingredient.

17. The method of claim 1, wherein the distributing the distributable object to the subject comprises distributing the distributable object in a secured receptacle to the subject.

18. The method of claim 1, further comprising:
authenticating, via the one or more processors, first authorization information, wherein the placing of the determined first dosage of the ingestible ingredient onto the physical substrate occurs in response to the authentication of the first authorization information;
authenticating, via the one or more processors, second authentication information, wherein the distributing the distributable object: (i) occurs in response to the authentication of the second authentication information, and (ii) comprises distributing the distributable object into a secured receptacle;
authenticating, via one or more processors of the secured receptacle, third authentication information; and
in response to the authentication of the third authentication information, unlocking the secured receptacle to thereby allow extraction of the distributable object.

19. The method of claim 1, further comprising:
authenticating, via the one or more processors, first authorization information, wherein the placing of the determined first dosage of the ingestible ingredient onto the physical substrate occurs in response to the authentication of the first authorization information; and
authenticating, via the one or more processors, second authentication information, wherein the distributing the distributable object occurs in response to the authentication of the second authentication information.

20. The method of claim 1, further comprising:
authenticating, via the one or more processors, authorization information, wherein the placing of the determined first dosage of the ingestible ingredient onto the physical substrate occurs in response to the authentication of the authorization information; and
wherein the authentication information comprises biometric data, passcode data, and/or data from a mobile device of the subject.

21. The method of claim 1, further comprising:
identifying, via the one or more processors, a user profile of the subject;
determining, via the one or more processors, if a replenishment of a medication from the user profile is available; and
if the replenishment of the medication is available, distributing a second distributable object comprising the replenishment of the medication to the subject.

22. The method of claim 1, further comprising:
based on the evaluation, via the one or more processors, determining a second dosage of the ingestible ingredient; and
based on the determined second dosage, via the one or more processors, calculating a predicted optimal time for a refill of the ingestible ingredient.

23. The method of claim 1, wherein the determining of the first dosage comprises receiving, via the one or more processors, the first dosage amount from an external database.

24. The method of claim 1, wherein the determining of the first dosage comprises receiving, via the one or more processors, the first dosage amount from a computing device of the subject.

25. The method of claim 1, further comprising:
determining, via the one or more processors, a sequence of dosing events, wherein the first dosage corresponds to a first event of the sequence of dosing events; and
sequentially distributing subsequent distributable objects to the subject, wherein each distribution event of the sequential distribution: (i) occurs in response to receiving authorization based on receiving a previously distributed distributable object back from the subject, and (ii) is logged via the one or more processors.

26. The method of claim 1, further comprising loading a cartridge containing the ingestible ingredient into a medication dispenser apparatus, wherein the cartridge is configured such that, upon tampering, heat is released to deactivate the ingestible ingredient.

27. The method of claim 1, further comprising loading a cartridge containing the ingestible ingredient into a medication dispenser apparatus, wherein the cartridge is configured such that, upon tampering, heat is released to deactivate the ingestible ingredient, and wherein the heat is released by:
releasing the heat from a thermal battery of a spool of the cartridge; or
releasing the heat from a resistively heated element of the spool of the cartridge.

28. The method of claim 1, further comprising loading a cartridge containing the ingestible ingredient into a medication dispenser apparatus, wherein the cartridge is configured such that, upon tampering, gas or liquid is released to deactivate the ingestible ingredient.

29. A system for tracking consumption of an ingestible ingredient, the system comprising:
a distributor device comprising a housing, and a communication interface;
a dispenser device housed within the distributor device, the dispenser device being configured to place dosages of ingestible ingredients onto physical substrates; and
one or more processors configured to:
determine a first dosage of an ingestible ingredient to administer to a subject;
control the dispenser device to place the determined first dosage of the ingestible ingredient onto a physical substrate to create a distributable object;
control the distributor device to distribute the distributable object to the subject;
control the distributor device to receive at least a portion of the distributable object back from the subject;
evaluate the received at least a portion of the distributable object; and
log the evaluation of the received at least a portion of the distributable object.

30. The system of claim 29, further comprising a cartridge comprising a spool and a locking mechanism; and
wherein the locking mechanism is configured to:
prohibit the spool from rotating when the locking mechanism is in a locked position;
allow the spool to rotate when the locking mechanism is not in the locked position; and
change between the locked position and the unlocked position based on a received signal.

31. The system of claim 29, further comprising a cartridge comprising one or more cartridge processors configured to communicate:
a location of the cartridge;
a status of the cartridge;
an access history of the cartridge;
a temperature of the cartridge; or
identifying information of the cartridge.

32. The system of claim 29, further comprising a cartridge comprising: (i) a spool, and (i) a tape including the ingestible ingredient and a waxed paper backing.

33. The system of claim 29, further comprising a cartridge housing physical substrates with no ingestible ingredient thereon.

34. The system of claim 29, further comprising a cartridge housing physical substrates with an ingestible ingredient affixed thereon.

35. The system of claim 29, wherein the one or more processors are configured to control the distributor device to distribute the distributable object to the user by opening a door of the distributor device.

36. The system of claim 29, wherein the one or more processors are configured to simultaneously:
(i) rotate a spool of a cartridge to place the determined first dosage of the ingestible ingredient onto the physical substrate to create the distributable object; and
(ii) open a door of the distributor device to distribute the distributable object to the subject.

37. The system of claim 29, wherein dispenser device is further configured to allow a user to replace a first cartage with a second cartridge, and wherein the second cartridge comprises the ingestible ingredient.

38. The system of claim 29, wherein the dispenser device is further configured to, if an amount of the ingestible ingredient in the dispenser device falls below a predetermined level:
provide an alert indicating that the amount of ingestible ingredient in the dispenser device is low; and
unlock a first cartridge from the dispenser device, thereby allowing the first cartridge to be removed and replaced with a second cartridge.

39. The system of claim 29, wherein the one or more processors are further configured to send data of the evaluation to a database.

40. The system of claim 29, wherein:
the one or more processors are further configured to open a door of the distributor device to allow the at least a portion of the distributable object to be deposited in the dispenser device;
the dispenser device further comprises a camera configured to capture a visual image of the at least a portion of the distributable object; and
the one or more processors are further configured to input data of the visual image into a machine learning algorithm to determine a remaining amount of the first dosage.

41. The system of claim 29, wherein:
the one or more processors are further configured to open a door of the distributor device to allow the at least a portion of the distributable object to be deposited in the dispenser device;
the dispenser device further comprises an ultraviolet (UV) camera configured to capture an UV image of the at least a portion of the distributable object; and
the one or more processors are further configured to input data of the UV image into a machine learning algorithm to determine a remaining amount of the first dosage.

42. The system of claim 29, wherein:
the one or more processors are further configured to open a door of the distributor device to allow the at least a portion of the distributable object to be deposited in the dispenser device;
the dispenser device further comprises a scale configured to weigh the at least a portion of the distributable object; and
the one or more processors are further configured to evaluate the received at least a portion of the distributable object by determining a remaining amount of the ingestible ingredient based on weight information received from the scale.

43. A system for tracking consumption of an ingestible ingredient, the system comprising:
a plurality of warehouses;
a plurality of vehicles;
a distributor device comprising a housing, and a communication interface;
a dispenser device housed within the distributor device, the dispenser device being configured to place dosages of ingestible ingredients onto physical substrates; and
one or more processors configured to:
determine a first dosage of an ingestible ingredient to administer to a subject;
control the dispenser device to place the determined first dosage of the ingestible ingredient onto a physical substrate to create a distributable object;
control the distributor device to distribute the distributable object to the subject;
control the distributor device to receive at least a portion of the distributable object back from the subject;
evaluate the received at least a portion of the distributable object; and
log the evaluation of the received at least a portion of the distributable object.

44. A method for tracking consumption of an ingestible ingredient, the method comprising:
determining, via one or more processors, a sequence of dosing events;
distributing a first distributable object to a subject, the first distributable object including a first dosage of an ingestible ingredient specified by a first dosing event of the sequence of dosing events; and
sequentially distributing subsequent distributable objects to the subject, wherein each distribution event of the sequential distribution: (i) occurs in response to receiving authorization based on receiving a previously distributed distributable object back from the subject, and (ii) is logged via the one or more processors.

45. The method of claim 44, further comprising receiving, via the one or more processors, an updated sequence of dosing events from an external system, the external system being a physician system, a medicinal decisional support system, a hospital system, or an assisted living facility system; and
wherein distribution events of the sequential distribution are done according to the updated sequence of dosing events.

46. The method of claim 44, further comprising:
identifying, via the one or more processors, a user profile of the subject including subject information of the user, wherein the subject information includes: an age of the subject, a gender of the subject, a weight of the subject, and/or a height of the subject; and
wherein the sequential distribution further includes logging each distribution event to the user profile.

47. The method of claim 44, further comprising:
receiving used distributable objects back from the subject;
evaluating the received used distributable objects; and
updating the sequence of dosing events based on the evaluations of the used distributable object; and
wherein the sequential distribution of the subsequent distributable objects is done according to the updated sequence of dosing events.

48. The method of claim 44, wherein the distribution events of the sequential distribution are logged to a distributed ledger.
